(19) United States
(12) Patent Application Publication
(10) Pub. No.: US 2024/0341938 A1
(43) Pub. Date: Oct. 17, 2024

(54) HOLDER FOR COMBINATION INJECTOR AND SPRAY DEVICE

(71) Applicant: pHi-Tech Animal Health Technologies, Ltd., Airport City (IL)

(72) Inventors: Gershon Goldenberg, Pardes Hanna-Karkur (IL); Udi Ashash, Sede Nahum (IL)

(73) Assignee: pHi-Tech Animal Health Technologies, Ltd., Airport City (IL)

(21) Appl. No.: 18/752,070

(22) Filed: Jun. 24, 2024

Related U.S. Application Data
(63) Continuation of application No. PCT/IB2022/062746, filed on Dec. 23, 2022.
(60) Provisional application No. 63/294,309, filed on Dec. 28, 2021.

(51) Int. Cl. A61D 7/00; A01K 37/00

(52) U.S. Cl. CPC A61D 7/00; A01K 37/00; A61M 2205/106

(57) ABSTRACT

A holder for a medicament administration device can include a base member, a movable member disposed on the base member such that the movable member can move relative to the base member between a first position and a second position, and two or more clamp members. Each clamp member can be coupled to the base member and to the movable member, and can be pivotable between a closed position corresponding with the first position of the movable member and an open position corresponding with the second position of the movable member. When in the closed position the clamp members can engage the head of a subject to restrain the subject against movement relative to the holder.

### CROSS-REFERENCE TO RELATED APPLICATIONS

The present application is a continuation of International Application No. PCT/IB2022/062746, filed Dec. 23, 2022, which claims the benefit of and priority to the earlier filing date of U.S. Provisional Application No. 63/294,309, filed on Dec. 28, 2021; each of these prior applications is incorporated herein by reference in its entirety.

### FIELD

The present disclosure concerns embodiments of an administration device for administering one or more medicaments to subjects, particularly livestock animals, and more particularly concerns automated administration devices for injecting a large number of feed or companion animals (e.g., poultry, swine, cattle, sheep, goats, ungulates, cats, dogs, and/or aquatic species including fish) with one or more medicaments.

### BACKGROUND

In the livestock industry, animals often must be administered substances such as medications or vaccines, for a variety of reasons. Typically, each producer must treat large numbers of animals. Treatment can often require injecting each animal with a plurality of substances, generally in liquid form. Such substances can include medicines, vaccines, hormones, food supplements and the like (hereinafter referred to generally as “medicament”). Administering such medicaments typically includes using an administration device, such as a syringe or drenching unit, from which a medicament dose is either manually or automatically administered to the animal.

Different medicaments must be administered at different points in an animal's life cycle. For example, in the poultry industry some are administered via in-ovo injections, some are administered immediately post-hatching, and some are administered later in the lifecycle of the chick. However, it can be difficult to efficiently administer substances to large numbers of chicks while ensuring that the chicks are handled with care and the medicament is administered correctly. Accordingly, there is a continuing need for improved medicament administration devices and methods for their use.

### SUMMARY

Described herein are embodiments of an administration device for administering one or more medicaments to a subject such as a feed animal (e.g., poultry, swine, cattle, sheep, goats, ungulates, cats, dogs, and/or aquatic species including fish). Certain disclosed embodiments are particularly directed to administering medicaments to day-old-chicks (DOCs). The administration device can be used to administer medicaments to a large number of DOCs in a quick and efficient manner to prevent or mitigate injection malfunction, spray malfunction, and/or to prevent or mitigate potential damage to the chicks.

In a representative embodiment, a holder for a medicament administration device can comprise a base member, a movable member disposed on the base member such that the movable member can move relative to the base member between a first position and a second position, and two or more clamp members each coupled to the base member and to the movable member. The clamp members being pivotable between a closed position corresponding with the first position of the movable member and an open position corresponding with the second position of the movable member, wherein when in the closed position the clamp members are configured to engage the head of a subject to restrain the subject against movement relative to the holder.

In some embodiments, when in the closed position the clamp members define a channel between them, the channel sized such that a subject's neck can be disposed within the channel. In some embodiments, the channel has a first end portion and a second end portion and a width of the channel tapers from a first, greater width at the first end portion to a second, narrower width at the second end portion.

In some embodiments, each clamp member is pivotably coupled to the base member via a respective pivot member extending through the clamp member and the base member. In some embodiments, the base member comprises one or more extension members each having an inner bore, and wherein each pivot member extends through the inner bore of at least one extension member.

In some embodiments, each clamp member is coupled to the movable member via a fastener extending through the clamp member and into a channel disposed in the movable member, and wherein movement of the movable member between the first and second positions causes corresponding movement of the fastener within the channel.

In some embodiments, the holder further comprises a biasing member configured to bias the clamp members into the closed position.

In some embodiments, wherein the base member comprises an extension portion defining a recess, the recess configured to receive and orient the subject's head when the subject is positioned within the holder. In some embodiments, the recess comprises a U-shape.

In some embodiments, the movable member comprises a main body and a tab extending from the main body at an angle.

In some embodiments, wherein the movable member comprises a U-shaped cutout.

In some embodiments, the holder further comprises one or more back supports configured to support a back of the subject to help restrain the subject against movement relative to the holder. In some embodiments, each back support is pivotable between an open position and a closed position.

In a representative embodiment, a method of using the holder can comprise positioning a subject such that the subject's neck is aligned with a first end portion of a channel defined by the two or more clamp members, and inserting the subject's neck into the channel such that the subject's head is restrained by the two or more clamp members, thereby restraining the subject against movement relative to the holder.

In some embodiments, the method can further comprise applying a force to the movable member causing the movable member to move from the first position to the second position, thereby pivoting the two or more clamp members into the open position such that the subject is released from the holder.

In some embodiments, the act of applying a force to the movable member comprises actuating a pneumatic piston, wherein a head portion of the piston contacts the movable member to provide the force.

In a representative embodiment, a medicament administration device can comprise a plurality of stations including, at least, a loading station configured to allow insertion of a subject into the administration device, and a release station configured to automatically release the subject from the administration device, and a carousel having a stationary portion on which the plurality of stations is disposed, and a movable portion that is movable relative to the stationary portion. The device can further comprise a holder coupled to the movable portion of the carousel and configured to engage a subject and rotate the subject between the plurality of stations. The holder can comprise a base member, a movable member disposed on the base member such that the movable member can move relative to the base member between a first position and a second position, and two or more clamp members each coupled to the base member and to the movable member. The clamp members can be pivotable between a closed position corresponding with the first position of the movable member and an open position corresponding with the second position of the movable member, wherein when in the closed position the clamp members are configured to engage the subject to restrain the subject against movement relative to the holder.

In some embodiments, the administration device can further comprise an injection station configured to automatically administer a medicament to one or more subjects using one or more injectors, and a spray station configured to automatically administer a medicament to one or more subjects using one or more spray nozzles.

In some embodiments, the loading station comprises a sensor configured to determine whether a hand of a user is present within the loading station.

In some embodiments, the holder further comprises one or more back supports configured to support a back of the subject to help restrain the subject against movement relative to the holder.

In some embodiments, the loading station and the release station each comprise an actuator configured to move a toothed linear member operatively engaged with the back support in order to pivot the back support between an open position and a closed position.

In some embodiments, when in the closed position the clamp members define a channel between them, the channel sized such that a subject's neck can be disposed within the channel. In some embodiments, the channel has a first end portion and a second end portion and a width of the channel tapers from a first, greater width at the first end portion to a second, narrower width at the second end portion.

In some embodiments, each clamp member is pivotably coupled to the base member via a respective pivot member extending through the clamp member and the base member. In some embodiments, the base member comprises one or more extension members each having an inner bore, and wherein each pivot member extends through the inner bore of at least one extension member.

In some embodiments, each clamp member is coupled to the movable member via a fastener extending through the clamp member and into a channel disposed in the movable member, and wherein movement of the movable member between the first and second positions causes corresponding movement of the fastener within the channel.

In some embodiments, the device further comprises a biasing member configured to bias the clamp members into the closed position.

In some embodiments, the base member comprises an extension portion defining a recess, the recess configured to receive and orient the subject's head when the subject is positioned within the holder.

In some embodiments, the movable member comprises a main body and a tab extending from the main body at an angle.

In some embodiments, the release station comprises an angled ramp portion configured to allow one or more subjects to slide out of the administration device.

In some embodiments, the release station comprises one or more guides pivotably connected to the administration device and configured to guide one or more subjects out of the administration device.

In some embodiments, the device further comprises one or more medicament containers fluidly coupled to the one or more injectors.

In some embodiments, the one or more subjects are chicks. In some embodiments, the chicks are day old chicks.

In some embodiments, the release station comprises one or more actuators configured to actuate the movable member. In some embodiments, the actuators are pneumatic. In some embodiments, the one or more actuators are coupled to the stationary portion. In some embodiments, the actuators comprise a piston having a head portion configured to contact the movable member to actuate the movable member from the first position to the second position.

In a representative embodiment, an administration device can comprise a housing, and a carousel disposed within the housing. The carousel can comprise a stationary portion including (a) a loading station, (b) an injecting station comprising one or more injectors, (c) a spraying station comprising one or more spray nozzles, and (d) a release station, and a movable portion rotatable relative to the stationary portion, the movable portion comprising first and second holders configured to retain first and second subjects. The device can further comprise a holder coupled to the movable portion of the carousel and configured to engage a subject and rotate the subject between the plurality of stations. The holder can comprise a base member, a movable member disposed on the base member such that the movable member can move relative to the base member between a first position and a second position, and two or more clamp members each coupled to the base member and to the movable member, the clamp members being pivotable between a closed position corresponding with the first position of the movable member and an open position corresponding with the second position of the movable member, wherein when in the closed position the clamp members are configured to engage the subject to restrain the subject against movement relative to the holder. The device can further comprise a dosing system comprising one or more pumps each comprising a piston, each pump being coupled to at least one of the injecting station and the spraying station, andone or more actuators each coupled to one or more pumps and configured to actuate one or more pumps.

In some embodiments, the one or more actuators are pneumatic actuators.

In a representative embodiment, a method can comprise providing any of the previously-described administration devices and using the administration device.

In some embodiments, using the administration device comprises inserting a subject into the holder by inserting the subject's neck between a first set of the two or more clamp members, rotating the subject to the injection station, and inserting a needle coupled to the injector to a selected depth within the subject. The method can further comprise automatically administering a dose of medicament when the needle reaches the selected depth, rotating the subject to the spraying station, and automatically administering a dose of medicament to the subject via the one or more spray nozzles.

In some embodiments, the method can further comprise rotating the subject to the release station, and actuating one or more actuators to engage the movable member such that the movable member moves from the first position to the second position thereby moving the clamp members from the closed position to the open position and releasing the subject.

In some embodiments, the method further comprises actuating one or more back support members from an open position to a closed position such that the one or more back supports help restrain the subject against movement relative to the holder.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a perspective view of an administration device, according to one disclosed embodiment.

FIG. 2 is a perspective view of the administration device of FIG. 1 with the housing shown partially transparent.

FIG. 3 is a top plan view of the carousel of the administration device of FIG. 1 without the housing.

FIG. 4 is a perspective view of the administration device of FIG. 1.

FIG. 5 is a perspective view of an administration device with the housing removed, according to another disclosed embodiment.

FIG. 6 is a perspective view of the administration device of FIG. 5.

FIG. 7 is an enlarged view of a portion of the administration device of FIG. 5.

FIG. 8 is a perspective view of a holder for a disclosed embodiment of an administration device.

FIG. 9 is a side elevational view of an administration device, according to another disclosed embodiment.

FIG. 10 is a perspective view of an exemplary embodiment of an injection station.

FIG. 11 is a side elevational view of the injection station of FIG. 10.

FIG. 12 is a perspective view of the injection station of FIG. 10.

FIG. 13 is a perspective view of an exemplary embodiment of an injector.

FIG. 14 is a perspective view of an exemplary embodiment of an injection station.

FIG. 15 is an enlarged perspective view of a portion of the injection station of FIG. 14.

FIG. 16 is a perspective view of an exemplary embodiment of a spraying station.

FIG. 17 is a perspective view of the spraying station of FIG. 16.

FIG. 18 is a perspective view of a portion of the spraying station of FIG. 16.

FIG. 19 is a side elevational view of a portion of the spraying station of FIG. 16.

FIG. 20 is a cross-sectional view of an exemplary embodiment of a pump for use in an administration device.

FIG. 21 is a cross-sectional view of the pump of FIG. 20.

FIG. 22 is an exploded perspective view of the pump of FIG. 20.

FIG. 23 is a perspective view of an exemplary embodiment of a dosing system for an administration device.

FIG. 24 is an enlarged perspective view of a portion of the dosing system of FIG. 23.

FIG. 25 is an enlarged perspective view of a portion of the dosing system of FIG. 23, with the cover portion shown transparently.

FIG. 26 is a perspective view of the dosing system of FIG. 23, with the cover portion shown transparently.

FIG. 27 is a perspective view of a portion of the dosing system of FIG. 23.

FIG. 28 is a side elevational view of an exemplary embodiment of a calibration device for use with an administration system.

FIG. 29 is a perspective view of an exemplary embodiment of a release station.

FIG. 30 is another perspective view of the spraying station of FIG. 16.

FIG. 31 is a perspective view of another exemplary embodiment of a release station.

FIG. 32 is a representative diagram of an exemplary computing environment.

FIG. 33 is an exemplary embodiment of a disclosed graphical user interface.

FIG. 34 is a top plan view of a portion of an exemplary embodiment of a dosing system including a pneumatic actuator for an administration device.

FIG. 35 is a perspective view of an exemplary embodiment of a dosing system including a pneumatic actuator for an administration device.

FIG. 36 is a top plan view of the dosing system of FIG. 35.

FIG. 37 is a perspective view of an administration device, according to another disclosed embodiment.

FIG. 38 is a top plan view of the carousel of the administration device of FIG. 37 without the housing.

FIG. 39 is a perspective view of the administration device of FIG. 37.

FIG. 40 is a perspective view of the administration device of FIG. 37 with the housing removed.

FIG. 41 is an enlarged view of a portion of the administration device of FIG. 37.

FIG. 42 is a perspective view of a holder for the administration device of FIG. 37.

FIG. 43 is a side elevational view of the administration device of FIG. 37.

FIGS. 44A-44B are side elevational views of a portion of the injection station of the administration device of FIG. 37.

FIG. 45 is a side elevational view of a portion of an exemplary injector.

FIG. 46 is a side elevational view of the injector of FIG. 45 being used on a subject.

FIG. 47 is a perspective view of a portion of the injection station of the administration device of FIG. 37.

FIG. 48 is a side elevational view of a portion of the spraying station of the administration device of FIG. 37.

FIG. 49 is a perspective view of a portion of the spraying station of the administration device of FIG. 37.

FIG. 50 is a perspective view of an exemplary dosing system that can be used with the administration device of FIG. 37.

FIG. 51 is a perspective view of the dosing system of FIG. 50.

FIG. 52 is a top down view of the dosing system of FIG. 50.

FIG. 53 is a cross-sectional view of a portion of the dosing system of FIG. 50.

FIG. 54 is a perspective view of a portion of the release station of the administration device of FIG. 37.

FIG. 55 is a side elevation view of a portion of the loading station of an exemplary administration device.

FIG. 56 is a top down view of an exemplary holder for the administration device of FIG. 55, shown in a closed position.

FIG. 57 is a top down view of the holder of FIG. 56, shown in an open position.

FIG. 58 is a perspective view of the holder of FIG. 56, shown with the clamp members removed.

FIGS. 59-60 are perspective views of a subject being inserted into the holder of FIG. 56.

FIG. 61 is a perspective view of a subject being released from the holder of FIG. 56.

FIG. 62 is a perspective view of the loading station of the administration device of FIG. 55, with a portion of the housing removed.

FIG. 63 is a perspective view of a portion of an exemplary release station for the administration device of FIG. 55.

FIG. 64 is a perspective view of a portion of an exemplary release station for the administration device of FIG. 55.

### DETAILED DESCRIPTION

### I. Definitions

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and system embodiment are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed apparatus and method embodiment can be used in conjunction with others. Additionally, the description may use terms like “provide” or “achieve.” These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.”

The term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is further away from the subject.

As used herein, the term “distal” refers to a position, direction, or portion of a device that is closer to the subject. Thus, for example, proximal motion of a device is motion of the device away from the subject, while distal motion of the device is motion of the device toward the subject.

The terms “longitudinal” and “axial” refer to an axis extending in the proximal and distal directions, unless otherwise expressly defined.

In the description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited. As used herein, the term “about” means the stated value and any value that still allows the device or feature to perform its intended function, and may include a variation within at least 10% of the stated value. For example, “about 100 degrees” means any value between at least 90-110 degrees, inclusive.

The term “medicament” as used herein refers to any substance that may be administered to a subject. Particular embodiments include, e.g., antibiotics, vaccines, hormones, food supplements, oils, vitamins, minerals, etc. In some embodiments, the medicaments are in liquid form. In other embodiments, the medicaments may be in powdered form and may be mixed with one or more solvents within the two or more containers or prior to being disposed therein. Exemplary medicaments include, but are not limited to: Nectiv® Forte, IC Quadro, Ornitin Triple, Salmin Plus, Gumbin® VP2, MB-1, Live Vector vaccines, Natrium ceftiofur, Amikacin, Gentaject, and combinations thereof. The term “subject” as used herein refers to a human or a non-human animal subject to a treatment, observation, or experiment.

The term “animal” can refer to a land animal, an aquatic animal, an avian, or an amphibian. For example, animals include but are not limited to: poultry, swine, cattle, sheep, goats, ungulates, cats, dogs, and/or aquatic species including fish. In some embodiments, the mammal is a bovine, equine, ovine, porcine, or caprine. A bovine may be a dairy animal or an animal raised for beef. Animals can include animals raised for human consumption or a domesticated animal. Examples of animals that can be fed and/or administered the disclosed combination include, but are not limited to: ruminant species, such as a sheep, goat, cow, heifer, bull, bullock, calf, ox, deer, bison, buffalo, elk, alpaca, camel or llama; ungulates, such as a horse, donkey, or pig; avians, such as chickens, including laying hens and broilers, turkey, goose, duck, Cornish game hen, quail, partridge, pheasant, guinea-fowl, ostrich, cmu, swan, or pigcon; aquatic animals, such as an aquaculture species, including fish (e.g., salmon, trout, tilapia, sea bream, carp, cod, halibut, snapper, herring, catfish, flounder, hake, smelt, anchovy, lingcod, moi, perch, orange roughy, bass, tuna, mahi, mackerel, cel, barracuda, marlin, Atlantic ocean perch, Nile perch, Arctic char, haddock, hoki, Alaskan Pollock, turbot, freshwater drum, walleye, skate, sturgeon, Dover sole, common sole, wolfish, sablefish, American shad, John Dory, grouper, monkfish, pompano, lake whitefish, tilefish, wahoo, cusk, bowfin, kingklip, opah, mako shark, swordfish, cobia, croaker, or hybrids thereof, and the like), crustaceans (e.g., lobster, shrimp, prawns, crab, krill, crayfish, barnacles, copepods, and the like), or mollusks (e.g., squid, octopus, abalone, conchs, rock snails, whelk, clams, oysters, mussels, cockles, and the like). Additionally, or alternatively, the animal may be a companion animal, such as canines; felines; rabbits; rodents, such as a rat, mouse, hamster, gerbil, guinea pig or chinchilla; birds, such as parrots, canaries, parakeets, finches, cockatoos, macaws, parakeets or cockatiel; reptiles, such as snakes, lizards, tortoises or turtles; fish; crustaceans; and amphibians, such as frogs, toads and newts.

The terms “food supplement,” “dietary supplement,” and “feed additive” as used herein refer to products intended to supplement the diet of a subject. Food supplements can include, but are not limited to, vitamins, fatty acids, direct fed microbials, probiotics, minerals, amino acids, enzymes, herbs and botanicals (including plant materials, algae, macroscopic fungi, and combinations thereof), and other substances.

### II. Exemplary Embodiments

Disclosed herein are embodiments of an administration device useable to administer one or more medicaments to one or more subjects (for example, Day-old chicks (DOCs)), either simultaneously or sequentially.

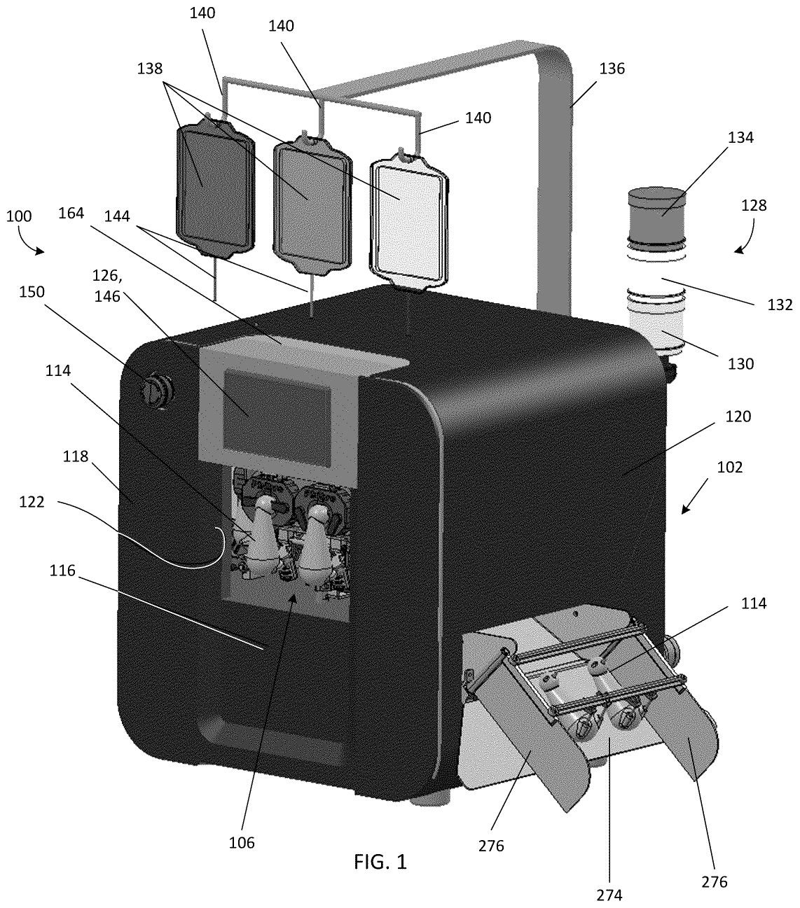

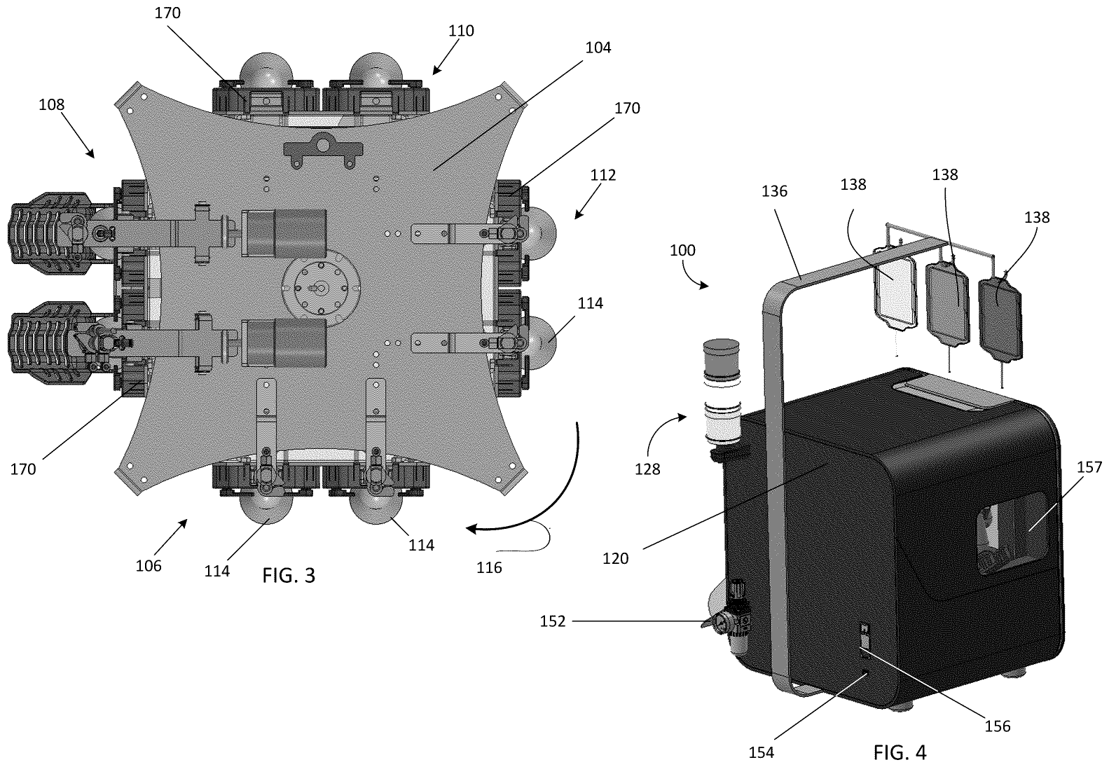

FIGS. 1-4 illustrate an exemplary administration device 100, according to one embodiment. As shown in FIG. 1, the administration device 100 can generally comprise a housing 102, a carousel 104 (see e.g., FIG. 3), and a plurality of stations each configured for a selected function. In the illustrated embodiment, as shown in FIG. 3, the administration device comprises four stations: a loading station 106, an injection station 108, a spray station 110, and a release station 112 (see e.g., FIG. 3). Subjects 114 can be loaded into the carousel 104 (e.g., manually or automatically) at the loading station 106. Once loaded, the carousel 104 rotates the subjects 114 through each station (e.g., in the direction of arrow 116) before automatically releasing the subjects at the releasing station 112. The administration device 100 can be configured to administer medicament to between 5000 and 7000 subjects per hour.

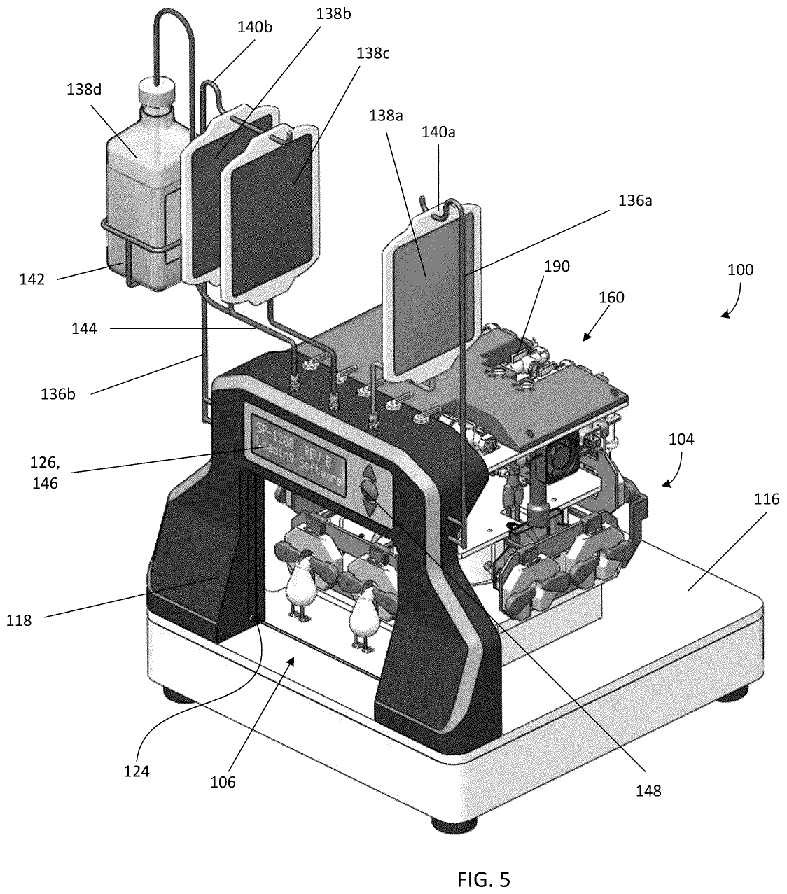

Referring to FIG. 1, the housing 102 can comprise a faceplate 118 and a cover 120. In the illustrated embodiment, the faceplate 118 comprises an opening 122, through which the loading station 106 is visible and through which the subjects 114 can be loaded onto the carousel 104. The subjects 114 can be manually loaded onto the carousel (e.g., by a user grabbing the subject and inserting the subject into the administration device), loaded onto the carousel automatically (e.g., the subjects can walk into the device 100 and the device can automatically restrain the subjects), or any combination thereof. In some embodiments, the opening 122 can comprise a sensor 124 (see e.g., FIG. 5) (such as a line of optical sensors) configured to determine whether a user's hand is within the opening. In such embodiments, the administration device 100 can be configured not to operate while a user's hand is sensed to prevent or mitigate injury to the user and/or the subjects 114.

Faceplate 118 can further comprise a control unit 126 including a display 146 configured to display information relating to the administration process. The control unit 126 can be used to control the medicament administration process and/or track data related to the administration process. For example, a user can input information about the administration process via control unit 126. In some embodiments, such as shown in FIG. 1, the display 146 can be a touch-screen display. In other embodiments, such as shown in FIG. 5, the control unit 126 can comprise one or more buttons 148 configured to allow a user to input information or scroll through displayed information. In still other embodiments, the administration device 100 can be controlled via a remote device (e.g., via an application or “app” such as for a smartphone, tablet, computer, etc.). In such embodiments, in lieu of or in addition to display 146, the remote device can comprise a remote device display.

In some embodiments, such as the embodiment shown in FIGS. 1-4, the administration device can comprise an indicator 128 (e.g., a light) configured to convey information about the status of device 100. For example, the indicator 128 can convey to a user whether the device is functioning normally, whether an alert and/or alarm has been triggered, and/or whether the device is stopped. In the illustrated embodiment, the indicator 128 is a light tower comprising a first indicator 130 indicating that the device is functioning normally, a second indicator 132 to indicate an alert and/or other issue (e.g., the device has stopped unexpectedly, the levels of medicament are low, the medicament temperature is out of limit, etc.), and a third indicator 134 indicating that the device is stopped and/or paused. However, in other embodiments, the indicator can be a single light configured to turn different colors and/or flash in different patterns. In still other embodiments, there can be an audible or text-based signal that conveys the same or additional information as the light indicators.

The administration device 100 can comprise one or more supports 136 configured to hold one or more medicament containers or medicament container holders 138. For example, in the illustrated embodiment, the administration device comprises one support 136. The support can comprise one or more hangers 140. The hangers can be configured to hold, for example, one or more medicament containers, such as flexible medicament containers 138. In some embodiments, each support and/or each hanger can comprise one or more sensors configured to detect whether a container is present, whether container is empty, etc.

In other embodiments, such as the embodiment shown in FIGS. 5-7, the administration device 100 can comprise two supports 136a and 136b. Support 136a can comprise a hanger 140a configured to hold at least one flexible medicament container, such as container 138a. Support 136b can comprise a hanger 140b configured to hold multiple medicament containers, such as flexible medicament containers 138b and 138c. Support 136b can further comprise a cup 142 configured to hold one or more rigid medicament containers, such as container 138d. However, in other embodiments, the cup(s) 142 can hold flexible medicament containers and the hangers 140 can hold rigid medicament containers. In still other embodiments, the administration device 100 can comprise any combination of supports, hangers, and cups as needed to hold the selected medicaments and medicament containers.

The medicament containers 138 can be coupled to the administration device 100 via one or more connecting tubes 144. The connecting tubes 144 can comprise portions of flexible material and/or portions of rigid material. For example, in the embodiment of FIGS. 5-7, the connecting tubes 144 comprise rigid portions where they couple to the administration device 100, thereby allowing multiple tubes to connect to the device without becoming entangled.

In some embodiments, as shown in FIG. 1, the administration device 100 can further comprise an emergency switch 150. In some embodiments, the emergency switch 150 can be configured to immediately stop the device 100 from functioning and/or return the device 100 to a default state. In other embodiments, the emergency switch can additionally automatically release all subjects 114 from the device.

Referring now to FIG. 4, the administration device 100 can further comprise an inlet valve 152, such as for pneumatic air, a power inlet 154, and a power switch 156. The inlet valve 152 is configured to allow the device 100 to be coupled to a pneumatic source. The power inlet is configured to allow the device to be coupled to a power source (e.g., an electrical outlet, batteries, a solar panel, USB, etc.), and the power switch 156 is configured to turn the device on or off.

As shown in FIG. 4, the cover 120 can comprise an opening 157 in a side panel thereof through which the injection station 108 can be accessed. The opening 157 can be sized such that a user can remove and/or replace the one or more injection needles through the opening without having to take apart the device 100.

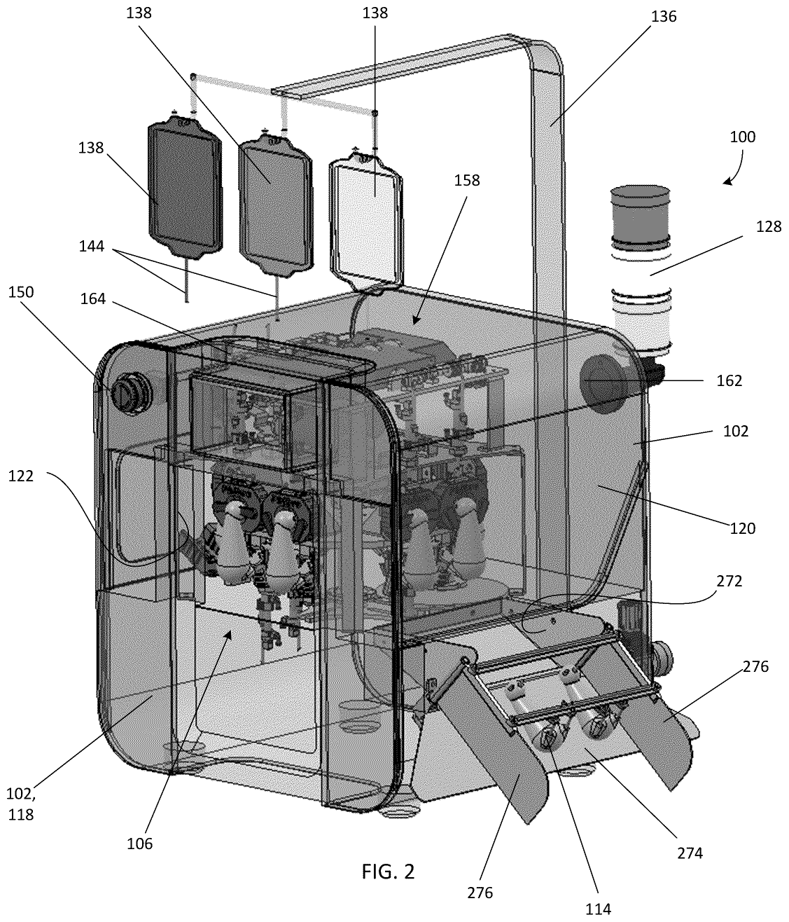

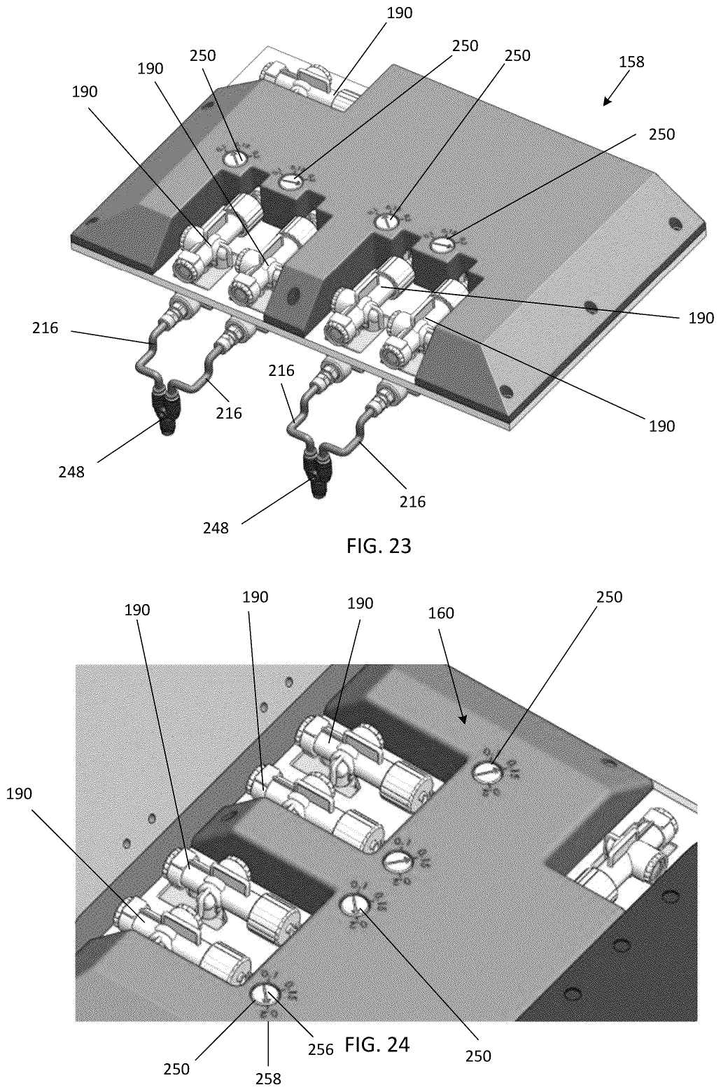

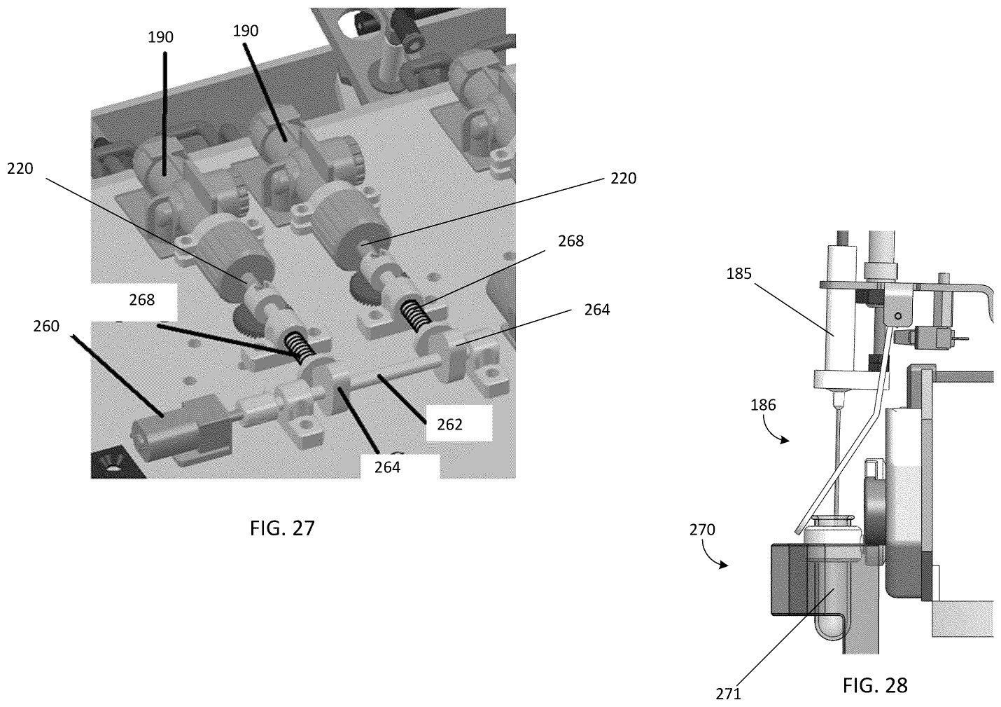

As mentioned previously, the administration device 100 can include a cover 120. The cover 120 can be used to protect the inner components of the administration device 100. For example, as shown in FIG. 2, the cover 120 can protect the dosing system 158, which can comprise a dosage adjustment system 160 (see e.g., FIGS. 23-27). In some embodiments, such as the illustrated embodiment of FIG. 2, the cover 120 can comprise a door 162 configured to allow a user to access the dosing system 158. In the illustrated embodiment, the door 162 is hingedly coupled to the cover 120 via hinges (not shown); however, in other embodiments the door can have any of various attachment mechanisms, for example, the door can be a sliding door. In the illustrated embodiment, the door 162 is the upper panel of the cover 120. However, in other embodiments, such as the embodiment shown in FIGS. 5-7, the door 162 can be a separate piece coupled to the upper panel (e.g., via hinges).

The door 162 can further comprise a handle 164 configured to allow a user to open the door 162 by gripping and pulling the handle. In some embodiments, the handle 164 can comprise a locking mechanism or other release that must be actuated before a user can open the door 162. In still other embodiments, the door 162 can comprise an automated locking mechanism that engages while the device 100 is in use and disengages automatically when the device is not in use (or e.g., when the device has been disconnected from the power source).

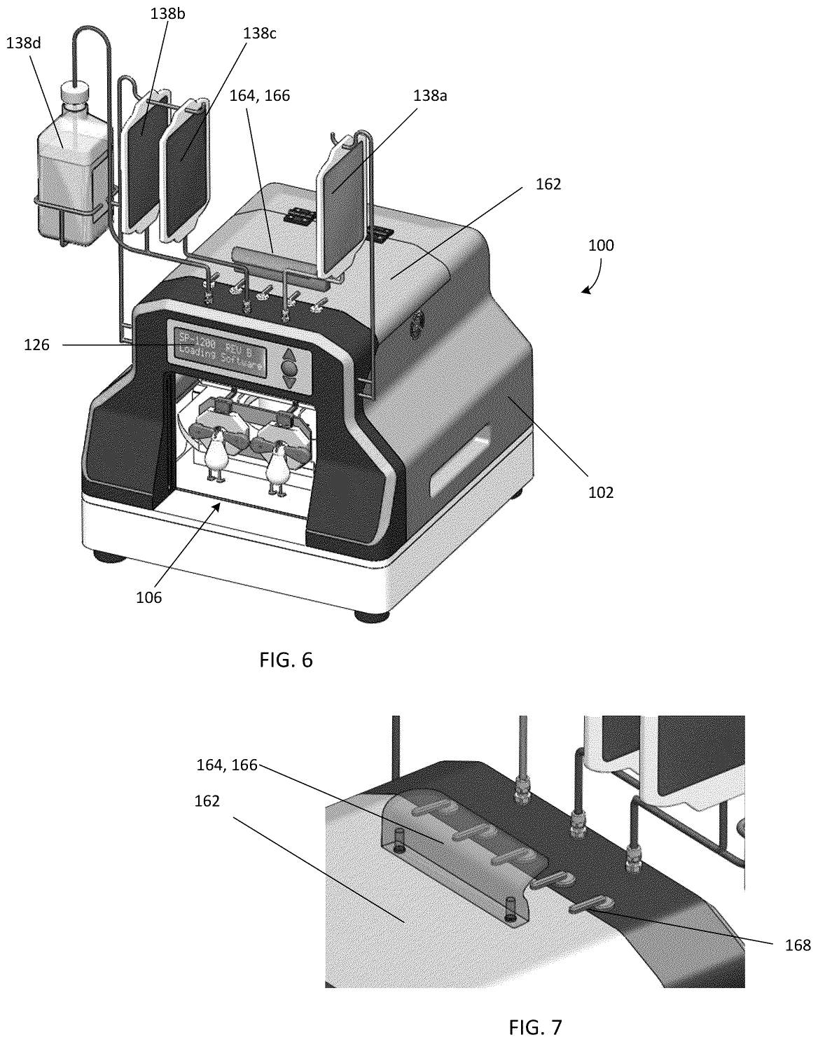

In some embodiments, as best shown in FIGS. 6-7, the door 162 can comprise an extension portion 166. In such embodiments, the extension portion 166 can serve as a handle 164 to open the door 162. As shown in FIG. 7, in some embodiments, the extension portion 166 can also serve as a shield for one or more valve shutoffs 168, preventing the valve shutoffs from being actuated accidentally. In the illustrated embodiment, the extension portion is substantially L-shaped, however, in other embodiments, the extension portion can have any of various shapes.

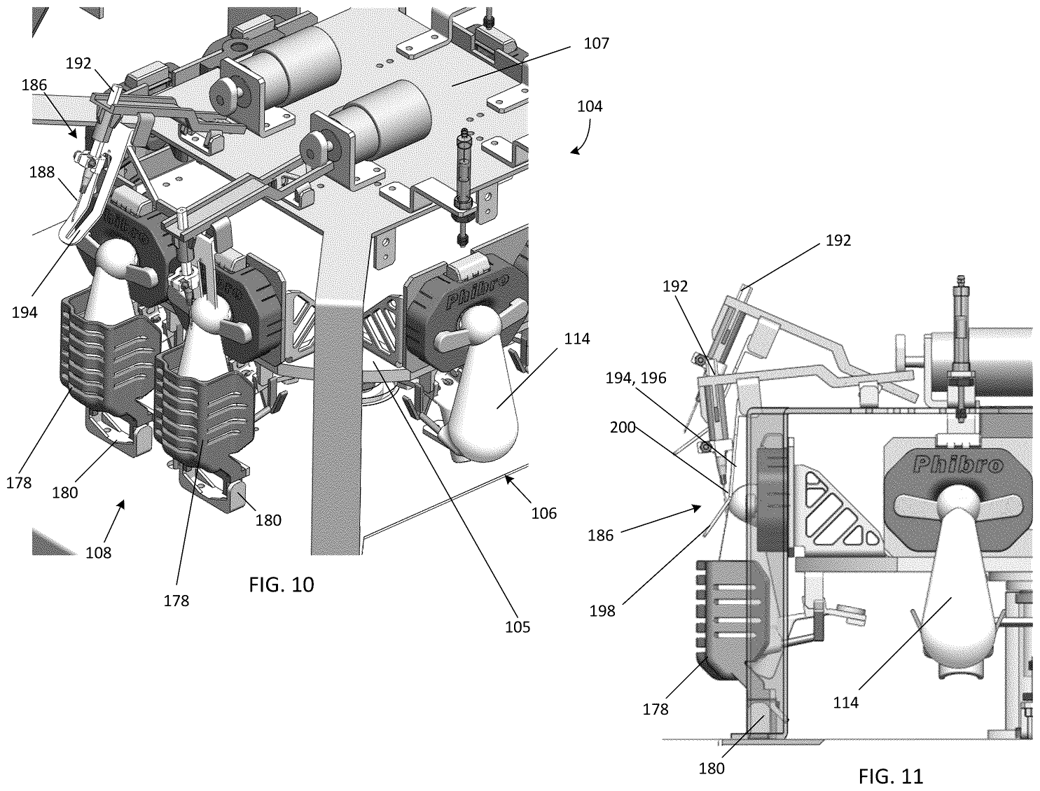

Referring now to FIG. 3, the carousel 104 can comprise one or more holders 170, for holding one or more subjects 114. In the illustrated embodiment, each holder 170 is configured to retain or hold two subjects 114. However, in other embodiments, the holders 170 can be configured to retain any number of subjects, for example, one subject, three subjects, four subjects, etc. The holders 170 can be coupled to the carousel 104 in a rotatable manner such that the holders 170 can rotate relative to the plurality of stations (e.g., stations 106, 108, 110, and 112). For example, as best shown in FIG. 10, the carousel 104 can comprise a movable portion 105 and a stationary portion 107 that is stationary relative to the movable portion 105. The holders 170 can be coupled to the movable portion 105 and the components of the plurality of stations (e.g., the injectors, the spray device, etc.) can be coupled to the stationary portion 107.

In the illustrated embodiments of FIGS. 1-6, the device 100 comprises four holders 170, such that each holder 170 can simultaneously be positioned at a respective station. However, in other embodiments, the device 100 can comprise any number of holders 170. For example, in a particular embodiment, the device can comprise five, six, seven, or eight holders, and further comprise a plurality of intermediate stations such that selected holders can be positioned at an intermediate station while other selected holders are positioned at one of the plurality of stations 106, 108, 110, 112.

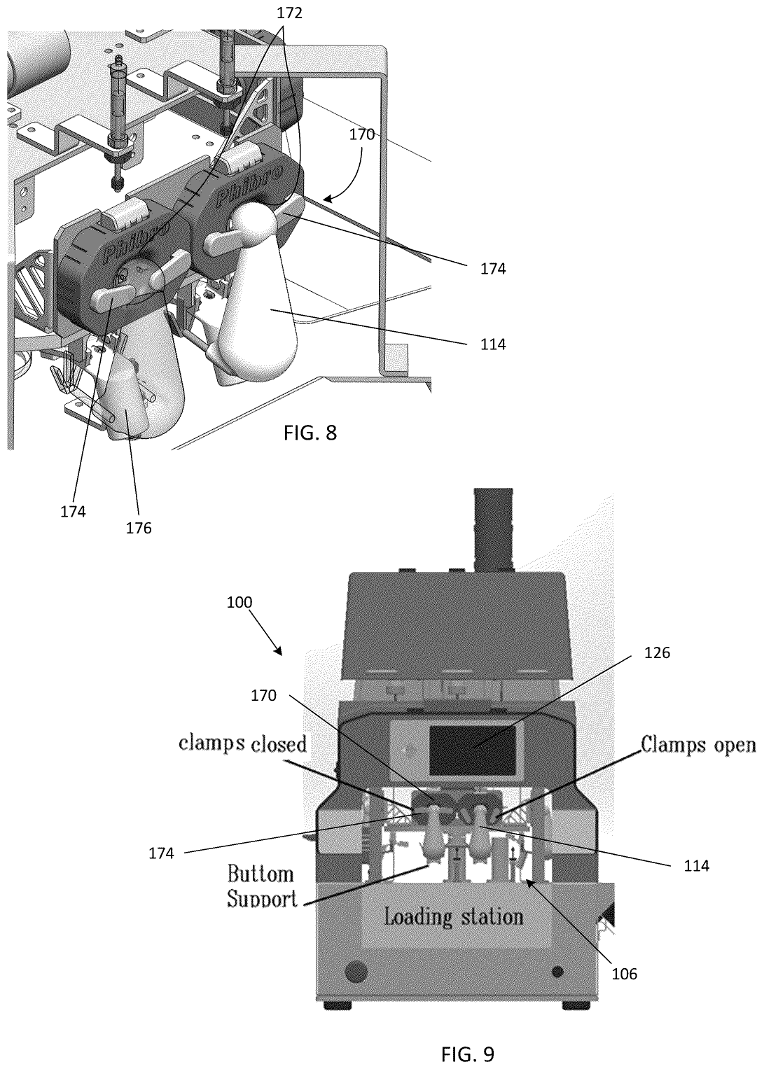

Referring now to FIG. 8, each holder 170 can comprise one or more openings 172 into which the subject's head can extend, and a pair of clamps 174 configured to restrain the subject 114 against movement relative the holder 170. The clamps 174 can have an open position (see FIG. 9), and a closed position (see FIGS. 8 and 9). During loading of the subject(s) 114 at the loading station 106, a user can insert the subject's head into the opening 172 and actuate the clamps 174. In some embodiments, the clamps 174 can be actuated manually (e.g., by pressing down on the clamps with, for example, a finger). In other embodiments, the administration device 100 can be configured to sense when a subject's head is within the opening 172 and to actuate the clamps 174 automatically to restrain the subject 114. In still other embodiments, a user can insert the subject's head into the opening 172 and actuate the clamps 174 by, for example, pressing a button on the control unit 126. In additional embodiments, the button can be located such that a user can press the button with a foot.

In some embodiments, as shown in FIG. 8, the holders 170 can further include a base support 176 configured to lift and/or restrain the body of the subject 114. For example, a subject 114 can be positioned such that when the subject's head is within the opening 172, the subject's midsection is supported by the base support. In other embodiments, the base support 176 can be configured to support, for example, the subject's feet, the subject's midsection, or both the subject's feet and midsection. In the illustrated embodiment, the base support 176 is an L-shaped support with a rounded front piece. However, in other embodiments, the base support 176 can have any of various shapes configured to hold the subject 114 in a selected position.

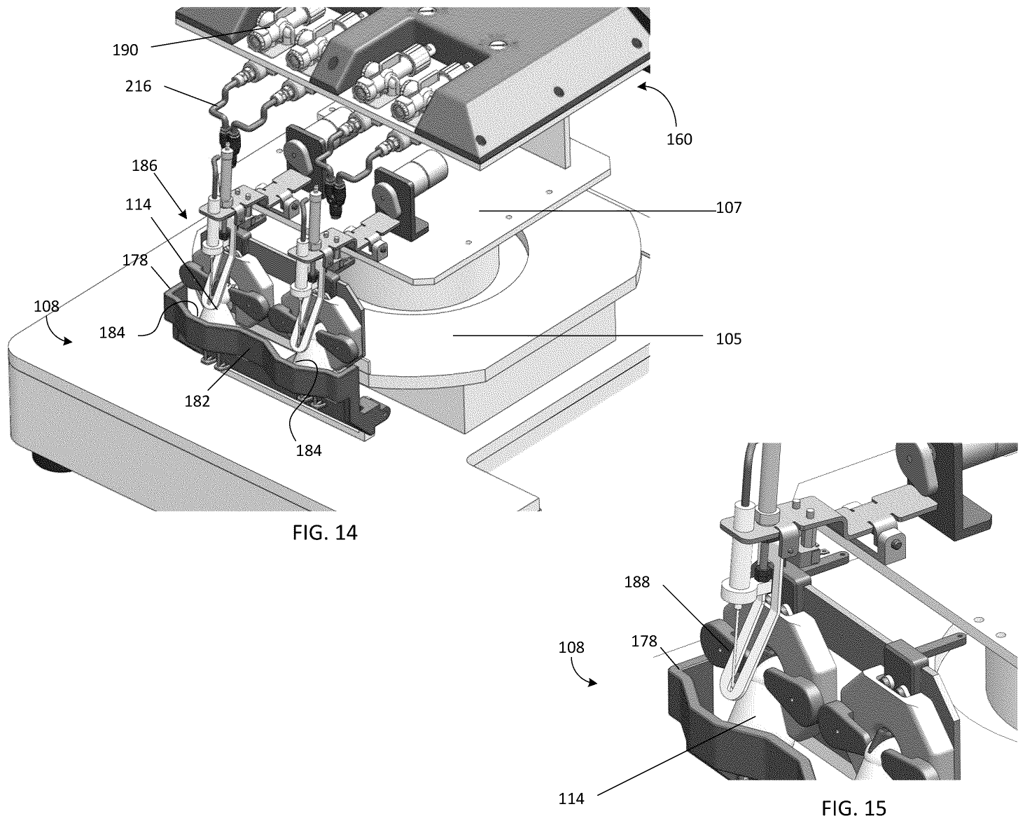

In some embodiments, different stations can comprise additional supports configured to additionally restrain the one or more subjects 114 carried by the holder 170. For example, as shown in FIG. 10, the injection station 108 can additionally comprise one or more back supports 178, configured to hold a respective subject in a selected position in order to facilitate injection. The back support 178 can configured to extend partially around a subject's back and hold the subject in a selected position. In the illustrated embodiment, the back support 178 is substantially C-shaped in cross-section. However, in other embodiments, the back support 178 can have any of various shapes configured to hold the subject in the selected position. For example, FIGS. 14-15 illustrate an alternative embodiment of a back support 178 comprising a horizontal bar 182 including a cutout 184 for each subject 114.

The back support 178 can be coupled to the platform using one or more hinges 180. The hinges 180 allow the back support 178 to move between an open position (see e.g., FIGS. 2 and 4) and a closed position (see e.g., FIG. 10). In some embodiments, the back support 178 can automatically move to the open position when the holder 170 is in motion and automatically move to the closed position when the holder 170 is stationary and/or when a sensor determines a subject is present.

Once the subjects 114 have been loaded into the holder 170 via the loading station 106, the movable portion 105 of the carousel 104 can rotate, moving the subjects from the loading station 106 to the injection station 108. In some embodiments, the loading station 106 can comprise a sensor configured to determine whether one or more subjects 114 have been loaded into the holder 170. In some embodiments, once at least one subject has been loaded, the administration device 100 will automatically rotate the holder 170 to the next station, for example, the injection station 108.

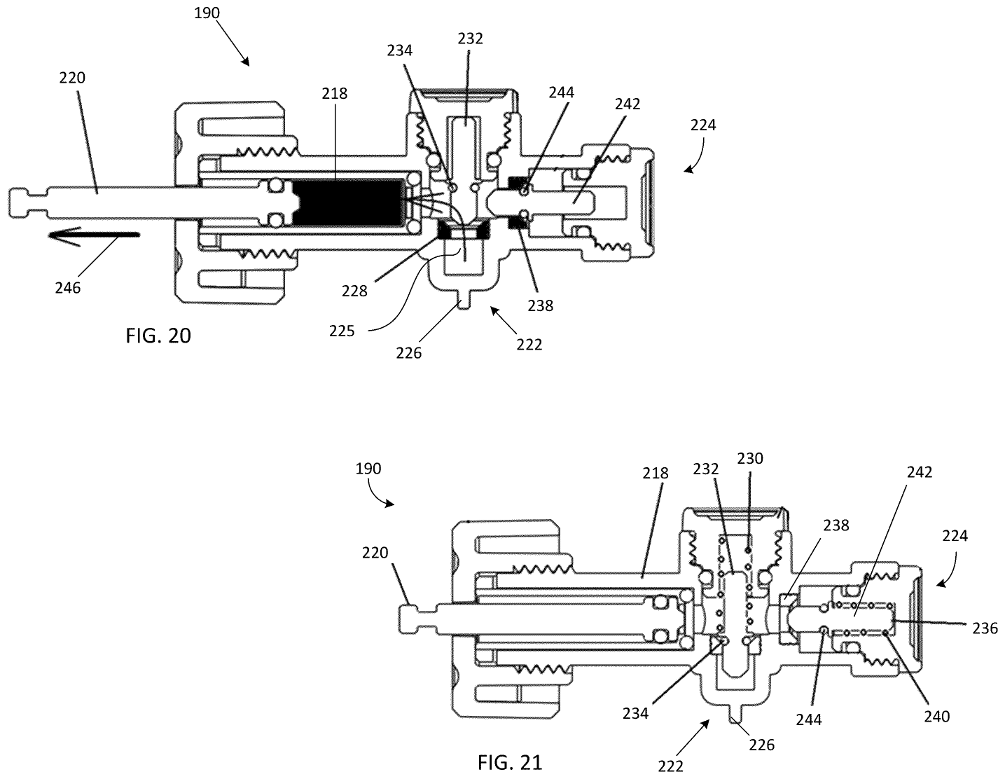

As shown in FIGS. 10-11, the injection station 108 can comprise one or more injectors 186 configured to administer a selected dosage of medicament to a subject 114. Each injector 186 can comprise a needle 188 fluidly coupled to one or more pumps 190 (see e.g., FIG. 20). The pumps 190 can be located, for example, in the dosing system 158, as described in more detail below. The needle 188 can be coupled to the one or more pumps 190 via one or more flexible tubes 216 (FIG. 23). The flexible tube(s) 216 can be coupled to an upper portion 192 of the injector 186. The needle 188 can be removably coupled to the injector 186 (e.g., using a luer lock), such that the needle 188 can be easily removed and/or replaced without disassembling the injector 186 and/or the administration device 100.

As shown in FIG. 10, the injector 186 can be pivotably coupled to the stationary portion 107 of the carousel 104 such that the injector 186 is movable between a ready position and a non-ready position. When the injector is in the ready position, if a subject 114 is present, the injector can rest against the subject such that a distal edge of the needle 188 can pierce the subject 114 at a selected location to a selected depth. In some embodiments, once the needle 188 has reached the selected injection depth, one or more doses of medicament can be administered automatically to the subject. When in the non-ready position, if a subject 114 is present, a distal edge of the needle 188 will not contact the subject.

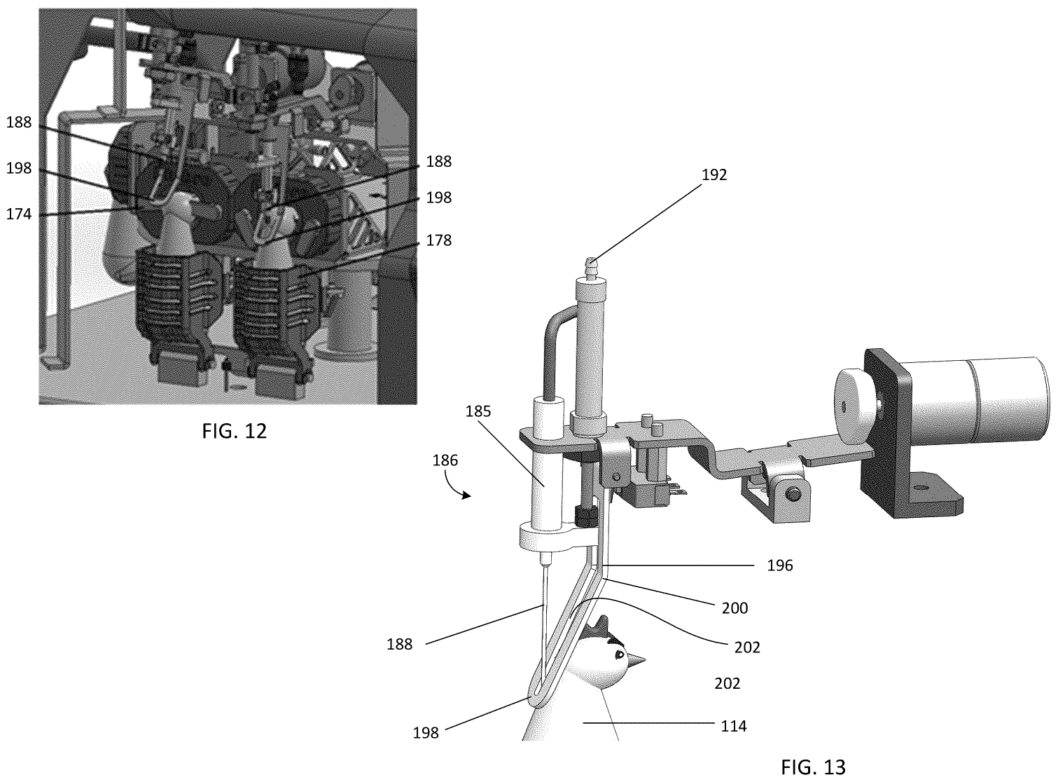

In some embodiments, the needle 188 is movable relative to the injector 186. For example, as shown in FIG. 13, the needle can be coupled to a pneumatic piston 185 configured to move the needle 188. Accordingly, the injector 186 can be movable between a ready position and a non-ready position, and the needle 188 can be movable between an extended position, in which the distal edge of the needle 188 pierces the subject 144, and a retracted position, in which the distal edge of the needle 188 does not contact the subject even if the injector 186 is in the ready position.

In the illustrated embodiment, each injector 186 further comprises a respective guide 194. As best seen in FIG. 13, the guide 194 can be an elongated member including a straight portion 196 and an angled portion 198 separated by a bend 200. The guide 194 can further comprise a slot 202, through which the needle 188 can extend. In some embodiments, as shown in FIG. 11, the guide 194 can be configured such that when the injector 186 is in the injecting position the bend 200 rests against the head and/or neck portion of the subject 114. This allows the injector 186 to be used for subcutaneous injections. Subcutaneous injections require that the needle penetrate the subject's skin but stop prior to penetration of the subject's muscle such that the medicament is deposited between the skin and the muscle. Such injections require that the needle 188 enter substantially parallel to the skin (see e.g., FIG. 11). As used herein, an object is “substantially parallel” with respect to a reference object or plane when the object is oriented at an angle of +20° or less with respect to the reference object or plane.

Once the needle 188 has reached a selected depth within the subject (e.g., as determined by a sensor), a pump 190 of the dosing system 158 (see e.g., FIG. 23) can actuate to automatically inject a dose of medicament. In embodiments wherein the needle 188 is coupled to two or more pumps 190, each dose of medicament can be injected either sequentially or simultaneously into the subject. In some particular embodiments, the injectors 186 can be configured to administer between 0.05 ml and 0.3 ml of medicament to each subject.

In some embodiments, the injection station 108 can further comprise one or more sensors configured to determine whether a subject is present. In some embodiments, the angled portion 198 of the injector can be configured as a sensor. In other embodiments, the base support 176 or back support 178 can be configured as a sensor. If a subject is not present, the injection station 108 can be configured to retain the needle 188 in the non-injecting position. Once the subject 114 has received the selected dose or doses of medicament and the injector 186 has moved out of the injecting position, the administration device 100 can automatically rotate the holder 170 to the next station, for example, the spraying station 110.

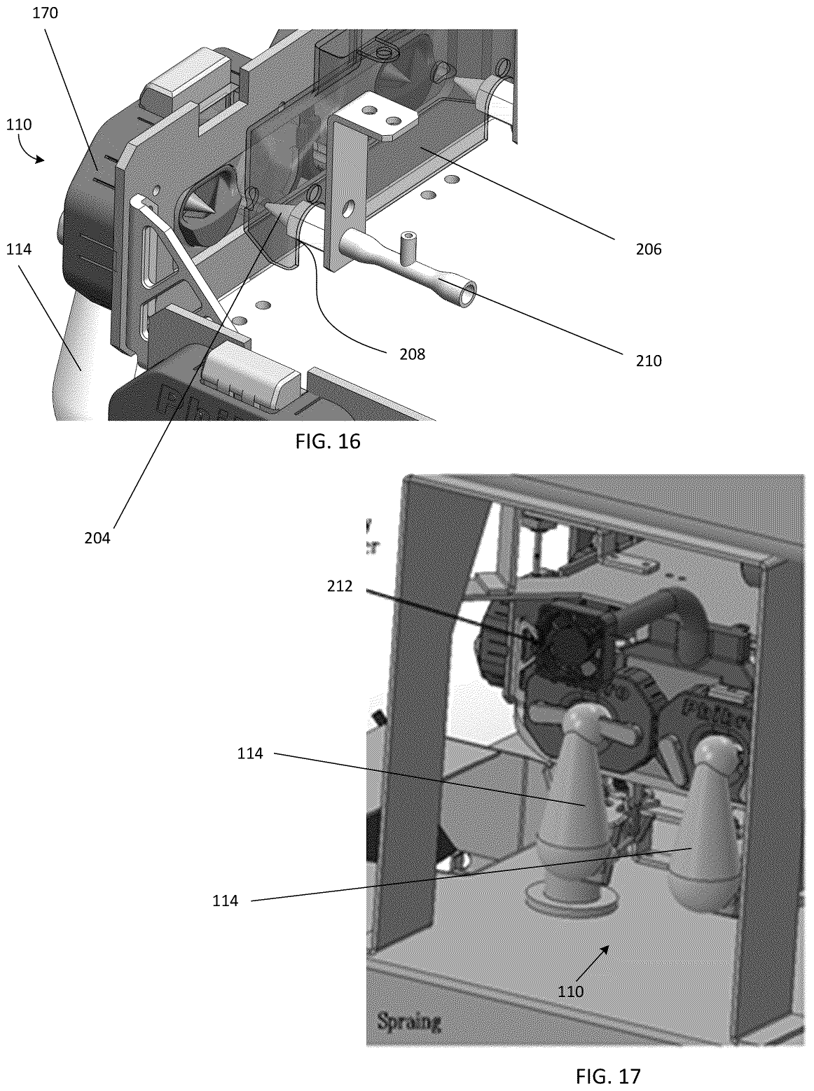

Referring now to FIGS. 16-19, the spraying station 110 can comprise one or more spray applicators or nozzles 204. Each spray nozzle 204 can be configured to administer a medicament in the form of a plurality of droplets to one or more mucosal tissues (e.g., the eyes, nose, and/or mouth) of the subject 114. In other words, the spray nozzle 204 can be configured to aerosolize the medicament. This form of delivery facilitates the development of both cellular and humoral immune reactions, and therefore protection from disease. In some particular embodiments, the spray nozzle 204 can be configured to administer 0.1 ml of medicament per subject.

Some live virus vaccines are too pathogenic for use as immunogens, whereas vaccinations with inactivated viruses may be less effective and produce undesirable side effects, such as inflammatory reactions at the site of the injection. Such vaccines can be delivered to the mucosa of a subject. Accordingly, in some embodiments, the spraying station 110 can be used to administer live vaccines to the mucosa of a subject, and the injection station 108 can be used to administer inactivated vaccines to a subject.

In certain embodiments, the administration device 100 is configured to allow a user to control and/or adjust the spread of the spray (distance and diameter). For example, the size of the spray droplets may be adjusted to beneficially affect delivery of a medicament in terms of medicament absorbance in the mucosal membrane. Accordingly, in certain embodiments, the spraying station 110 further comprises a mechanism for controlling and/or adjusting the size of the spray droplets. For example, the size of the droplets can be adjusted by changing the size of the spray nozzle 204 (e.g., by replacing the spray nozzle) and/or by controlling the pressure of the spray. In some embodiments, this spray droplet adjustment can be achieved by adjusting the pressure applied by the pump or the motor using a pulse-width modulation (PWM) controller. This allows differently sized droplets to be used on animals of different sizes and ages, for example, using smaller droplets on smaller and/or younger animals. In addition, this allows for the adjustment of droplet size for different medicaments and/or vaccination components having different viscosity, which can influence the size of the created droplets.

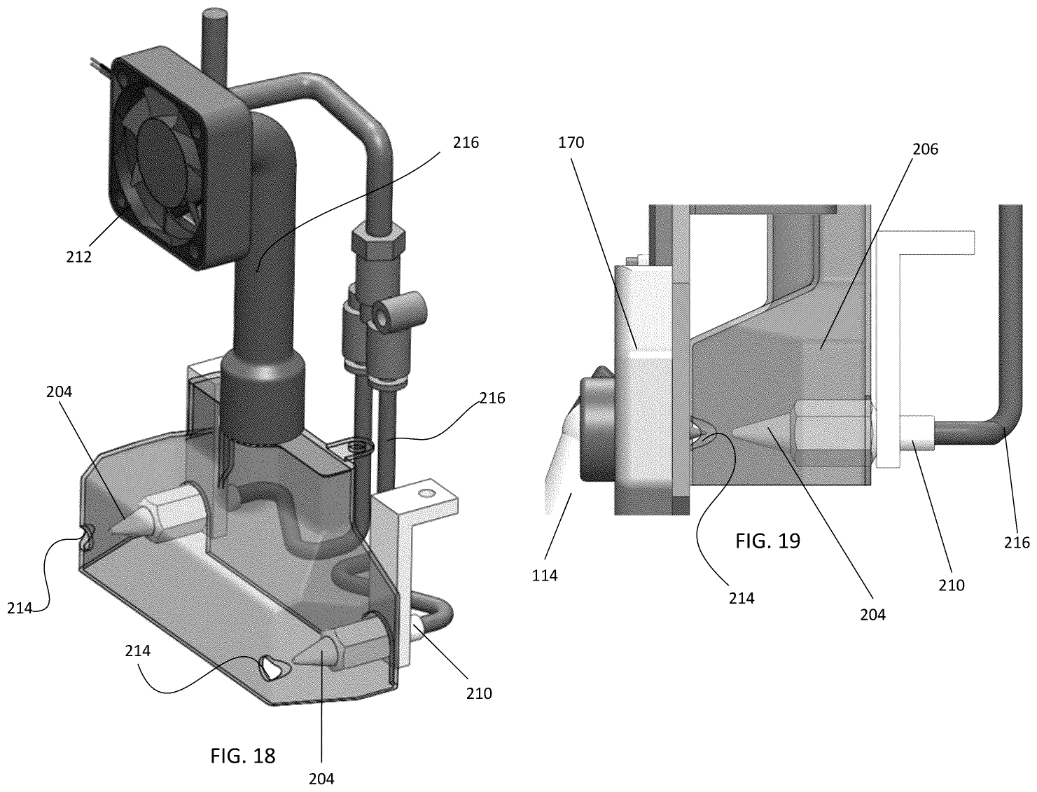

The spraying station 108 can further comprise a funnel or hood 206 configured to guide the sprayed medicament to the mucosal tissue of the subject 114. The hood 206 can further be configured to mitigate or prevent the aerosolized particles from entering the other areas of the administration device 100. The hood 206 can comprise one or more first openings 208 into which each spray nozzle 204 can extend and one or more second openings 214 (FIGS. 18 and 19) into which a portion of the subject's head can extend. Each spray nozzle 204 can comprise a connector 210 configured to couple one or more flexible tubes 216. The flexible tubes 216 can couple the spray nozzle to one or more pumps 190 (see e.g., FIG. 20). The pumps 190 can be located, for example, in the dosing system 158, as described in more detail below.

Referring now to FIG. 17, the spraying station can further comprise a ventilator/acrator/fan 212. The fan 212 can be configured to direct the aerosolized medicament out of the spraying station and into a filter (not shown). As shown in FIG. 18, the fan 212 can be coupled to the hood 206 via a tube 216.

Once the aerosolized medicament has been administered to the subject 114, the administration device 100 can automatically rotate the holder 170 to the next station, for example, the release station 112.

Referring now to FIGS. 20-27, as mentioned previously, each injector 186 and/or spray nozzle 204 can be coupled via one or more flexible tubes 216 to one or more pumps 190. The one or more pumps 190 and associated tubing can form a dosing system 158 which can be coupled to the one or more medicament containers 138 and through which the medicament can flow.

Each pump 190 can be a plunger or piston pump. In the illustrated embodiment of FIGS. 20-22, each pump 190 can include a dosing chamber 218, a piston 220, and one or more one-way valves (e.g., two valves 222 and 224). When actuated, pump 190 can pull medicament into the dosing chamber 218 through the first or inlet valve 222, as shown by arrow 225, and can then push the medicament from the dosing chamber 218 to the injector 186 and/or spray nozzle 204 through the second or outlet valve 224.

As shown in FIG. 20, the inlet valve 222 can comprise an inlet 226, an opening 228, a biasing member 230 (see FIG. 21), a stopper 232, and an O-ring 234 disposed around a first end portion of the stopper 232. The outlet valve 224 can comprise an outlet 236 (see FIG. 21), an opening 238, biasing member 240 (see FIG. 21), a stopper 242, and an O-ring 244 disposed around a first end portion of the stopper 242. Each O-ring 234, 244 can be sized to occlude a respective opening 228, 238 in combination with a respective stopper 232, 242.

In the illustrated embodiment, biasing members 230 and 240 are springs. In other embodiments, the biasing members can be, for example, compressible elastic sleeves. The biasing members 230, 240 can be configured to bias the stoppers 232, 242 into a first position. In the first position, each stopper 232, 242 can occlude a respective opening 228, 238, thereby preventing the flow of medicament through the opening 228, 238.

In the illustrated embodiment, each stopper 232, 242 has a cylindrical shape with a tapered first end portion. However, in other embodiments, each stopper can have any of various shapes, including, but not limited to, a sphere, a disc, a cone, a cube, a rectangle, a pyramid, a frustoconical shape, etc. In some embodiments, both stoppers 232, 242 can have the same shape, and in other embodiments, each stopper 232, 242 can have a different shape.

In use, the dosing chamber 218 can be filled in the following exemplary manner. The piston 220 can be moved backward (e.g., in the direction of arrow 246) relative to the dosing chamber 218. As the piston 220 moves backward, the pressure in the dosing chamber 218 is reduced. The reduced pressure within the dosing chamber 218 causes the first stopper 232 to move such that it no longer precludes fluid flow through the first opening 228, as shown in FIG. 20. Medicament can then flow through the inlet 226, through opening 228, and into the dosing chamber 218 until the chamber is filled. Once the dosing chamber 218 is filled with medicament, there is no longer a pressure differential within the dosing chamber and the biasing member 230 moves to the first position such that the stopper 232 and associated O-ring 234 occlude the opening 228 and further medicament is prevented from entering the chamber.

Once the dosing chamber 218 is filled, the medicament dose can be injected by moving the piston 220 forward, creating a sufficient positive pressure within the dosing chamber 218 to overcome the biasing force of the biasing member 240. This causes the biasing member 240 (and therefore the stopper 242) to retract to a second, or open position such that the stopper 242 no longer occludes the opening 238, as shown in FIG. 21. In the open position, the stopper 242 is moved axially such that at least a portion of the outlet 236 is unobstructed such that medicament can pass through it. The medicament can pass through the opening 238 and out of the pump 190 through the outlet 236. The positive pressure created by the piston 220 pushes the medicament through a connecting tube (such as connecting tube 216) through the needle and/or spray nozzle of the administration device, and into the subject.

In some embodiments, during injection of the medicament, the connecting tube (e.g., connecting tube 216) can expand slightly due to the positive pressure within the tube. Once the injection is complete and the pressure inside the tube is reduced, the tube contracts to its original size. The contraction of the tube to its original size can cause medicament to leak from the needle and/or spray nozzle. In such embodiments, when the biasing member 240 is in the second position, the stopper 242 partially obstructs or occludes outlet 236, thereby reducing the volume of outlet 236. The reduced volume of outlet 236 can correspond to the difference in volume between the expanded volume of the tube and the contracted volume of the tube. In this way, leakage of medicament from the needle and/or spray nozzle after an injection is completed can be eliminated or mitigated.

In other embodiments, the valves 222, 224 can be configured such that they can be actuated electrically (e.g., by a microprocessor) between the first and second configurations. In still other embodiments, the biasing members can be configured such that they can be actuated manually (e.g., by pressing a button, flipping a switch, or turning a lever), magnetically, hydraulically, and/or pneumatically.

Referring now to FIG. 23, in the illustrated embodiment, the dosing system 158 comprises five pumps 190; however, in other embodiments, the dosing system 158 can comprise a greater or fewer number of pumps 190. One or more of the pumps 190 can be coupled to the injectors 186, and one or more of the pumps 190 can be coupled to the spray nozzles 204. For example, in the illustrated embodiment of FIG. 23 each needle 188 can be coupled to two pumps 190 via one or more tubes 216, which allows administration of the same medicament by different pumps 190 to needle 188 or two different medicaments to needle 188. The tubes 216 can be coupled to a luer lock 248 configured to releasably couple the needle 188, as described previously, such that the needle 188 can easily be replaced without disassembling administration device 100. The remaining pump 190 can be coupled to the one or more spray nozzles 204. In other embodiments, the dosing system 158 can comprise any number of pumps 190 arranged in any configuration. For example, in some embodiments, each needle 188 is coupled to a single pump 190. In other embodiments, each spray nozzle 204 can be coupled to two or more pumps 190, or each spray nozzle 204 can be coupled to a single pump 190.

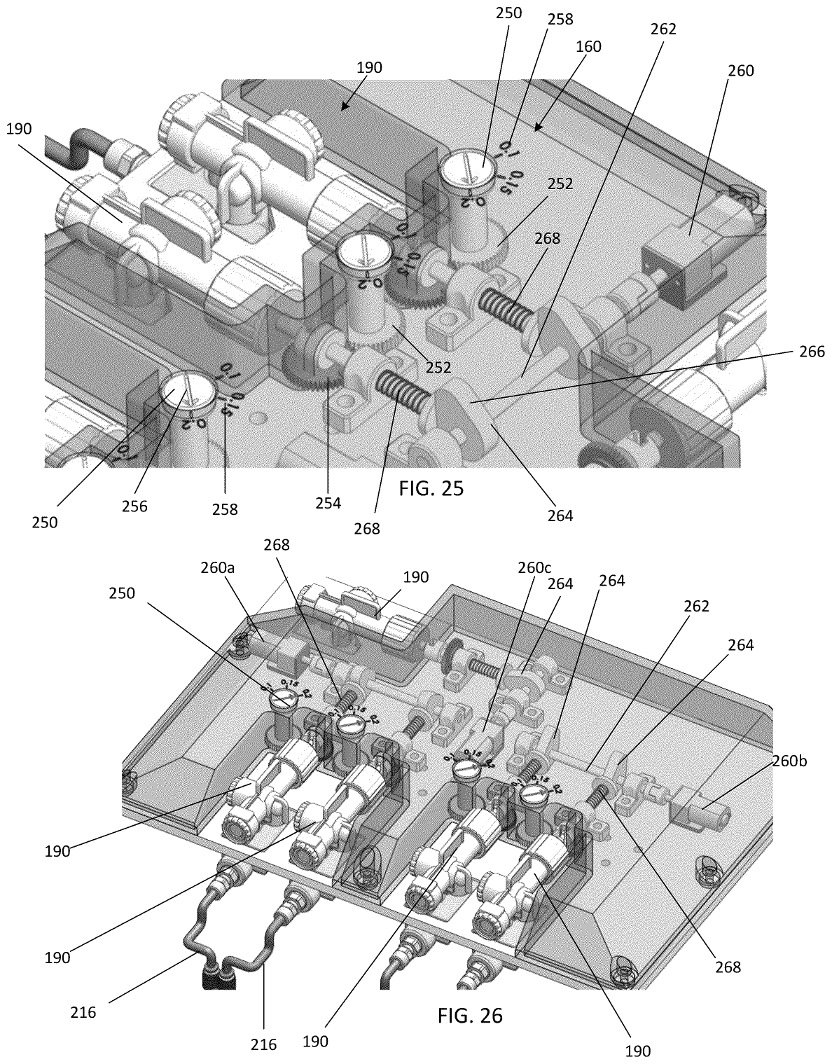

The administration device 100 can comprise a dosage adjustment system 160 configured to adjust the volume of medicament in each dose. Referring to FIG. 23, each pump 190 can comprise a respective dial 250 operatively coupled to the pump and configured to adjust the volume of the dosing chamber 218. A user can adjust the volume of medicament by, for example, turning a dial 250 of the dosage adjustment system 160. As shown in FIG. 25, the dial 250 can be coupled to a first toothed gear 252 configured to engage a second correspondingly toothed gear 254 coupled to the piston 220. A user can turn the dial 250 to adjust the position of the piston 220 within the dosing chamber 218, thereby adjusting the dosage volume of medicament. For example, a dosage can be between 0.1 ml and 0.3 ml. In some particular embodiments, a user can operate dial 250 to select a desired dose volume, such as a volume of from greater than 0 ml to at least 0.5 ml, such as dose volumes of 0.1 ml, 0.15 ml, 0.2 ml, 0.25 ml, 0.3 ml, 0.35 ml, 0.4 ml, 0.45 ml and 0.5 ml.

The dosage adjustment system 160 can comprise volume indicia 258 indicating the dose volumes that a user can select. The volume indicia 256 can be arranged around the dial 250. In some embodiments, each dial 250 can comprise a dial indicator 256 (e.g., an arrow) indicating the selected dosage volume by pointing to it. The dial can be adjusted using an electronic control system, such as control unit 126. In some embodiments, the dial 250 can be adjusted using a user's fingers. In other embodiments, a user may use a tool, such as a screwdriver, to adjust the dosage. In such embodiments, the dial 250 can comprise a channel or divot into which the tool can extend to actuate the dial. In still other embodiments, the dial 250 can be adjusted electronically.

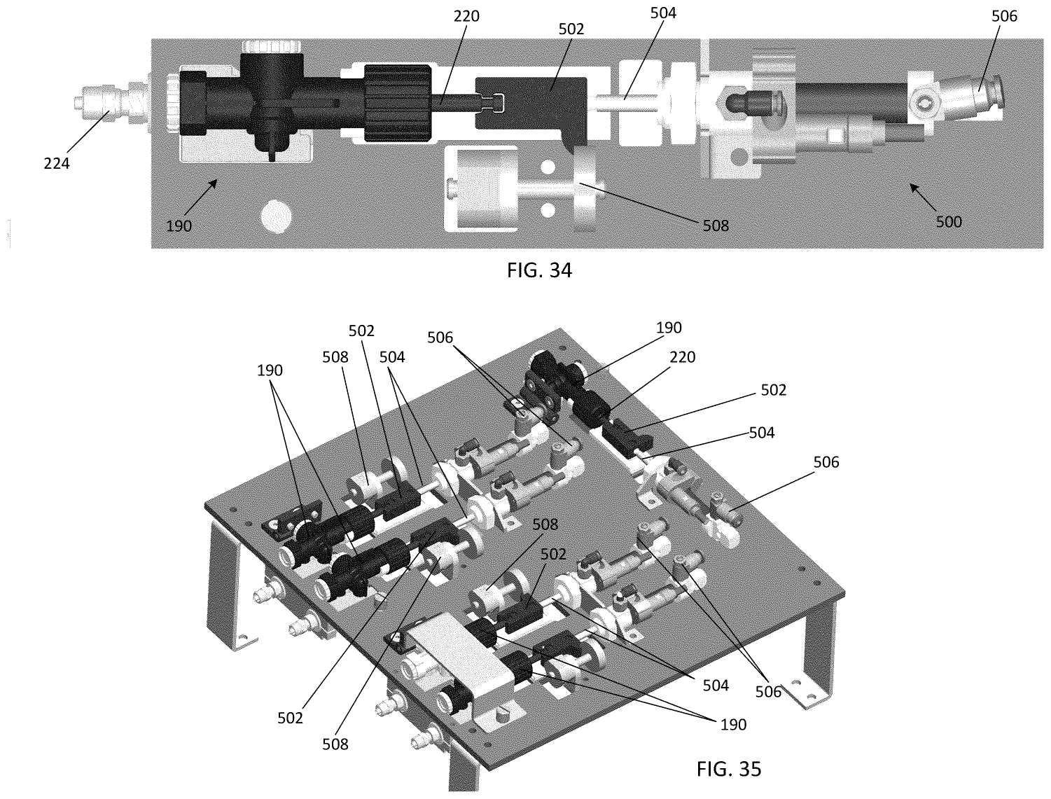

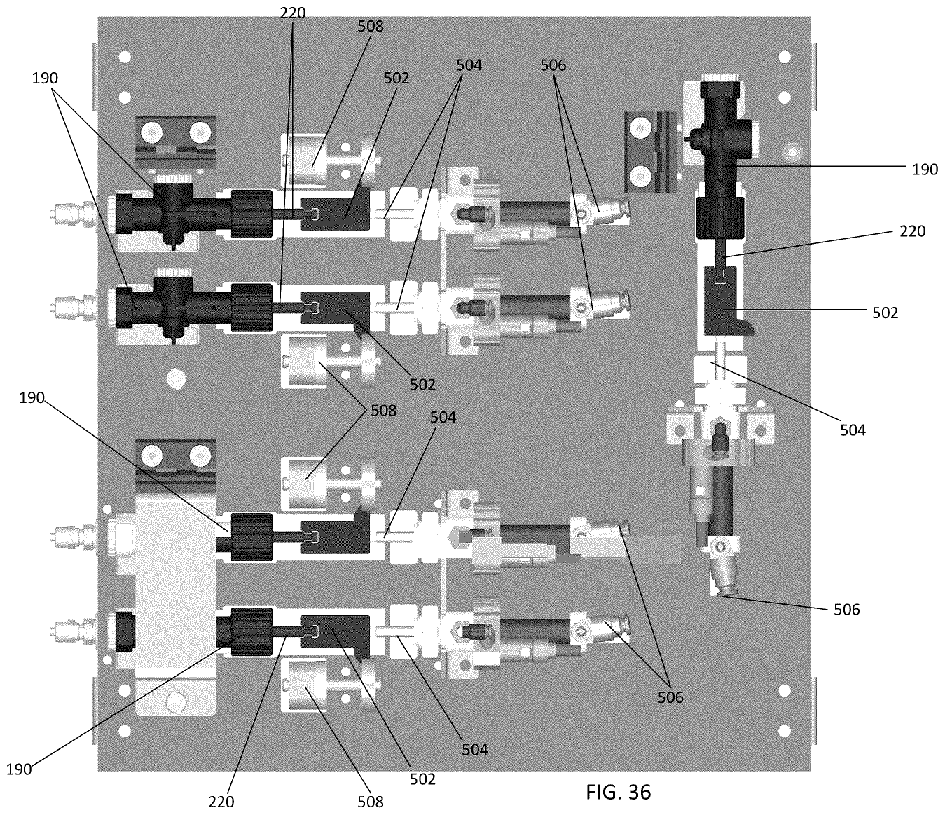

In some embodiments, such as the embodiment shown in FIGS. 34-36, each pump 190 can be actuated using a pneumatic actuator 500. The pneumatic actuator 500 can comprise an adapter 502, a pneumatic piston 504, and an air inlet 506. The adapter 502 can be configured to couple the pneumatic piston 504 to the pump piston 220 such that movement of the pneumatic piston 504 causes corresponding movement of the pump piston 220. Each air inlet 506 can be coupled (via, for example, a tube or hose) to a pneumatic source configured to actuate the pneumatic piston 504. Each pneumatic actuator 500 can further comprise a dial mechanism 508 configured to control the dose volume of a respective pump 190. For example, the dial mechanism 508 can control the position of the pump piston 220 relative to the pneumatic piston 504, thereby controlling the volume of medicament within the dosing chamber 218 (FIG. 20) of a respective pump 190.

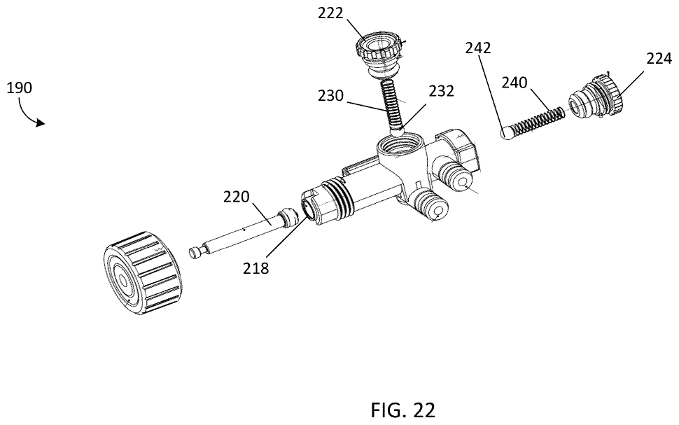

In other embodiments, such as the embodiment shown in FIGS. 25-27, each pump 190 can be actuated by a motor 260. The motor can, for example, be a DC motor. The motor 260 can actuate a drive shaft 262 operatively coupled to the piston 220. The drive shaft 262 can comprise one or more cam elements 264 each configured to apply an actuation force to the one or more pistons 220. In the illustrated embodiment of FIG. 26, each pump 190 has a respective cam element 264. However, in other embodiments, one or more pumps 190 can be actuated by the same cam element 264. The cam elements 264 can, for example, comprise an asymmetrical portion 266 (see e.g., FIG. 25) configured to rotate with the rotation of the drive shaft 262. As the drive shaft 262 turns, the asymmetrical portion 266 applies a force (e.g., a pushing force) to the piston 220 (scc e.g., FIG. 22), moving the piston 220 within the dosing chamber 218 and thereby administering a dose of medicament. Each piston 220 can further comprise a biasing member 268 (e.g., a spring) configured to bias the piston 220 against the cam element 264 and to return the piston 220 to a default position after actuation.

In some embodiments, one motor 260 can control one or more pumps 190. For example, referring to FIG. 26, the illustrated embodiment includes three motors 260a, 260b, 260c. The first and second motors 260a, 260b each control two pumps 190 via a single drive shaft 262, and the third motor 260c controls a single pump 190 via a drive shaft 262. In other embodiments, each motor 260 can control a respective pump 190. In still other embodiments, each motor 260 can control three or more pumps 190.

In other embodiments, each motor 260 can be coupled to a rotating gear that engages a plurality of corresponding teeth on the drive shaft 262 and/or the piston 220 in order to cause movement of the piston 220 within the dosing chamber 218. In such embodiments, the motor 260 can further comprise an encoder configured to, by controlling the rotation of the motor 260, set the position of the piston head 220 within the dosing chamber 218 and thereby control the volume of the medicament dose to be injected. This function can be used to set different medicament volumes to be injected.

Alternatively, the pumps 190 can be actuated using, for example, a hydraulic piston in lieu of the previously described embodiments. In such cases, the hydraulic piston can directly actuate the piston 220 of the pump 190 similarly to pneumatic actuator 500.

In some embodiments, the administration device 100 may require recalibration. For example, the device 100 may require recalibration prior to an initial use, between each batch, and/or after a predetermined number of injections and/or sprays. Accordingly, in certain embodiments, the control unit 126 can comprise a calibration system for calibrating the dosage of medicament administered by each pump 190.

Referring now to FIG. 28, the calibration system can function using one or more calibration units 270. The calibration units 270 can be separate components that can be releasably coupled to a respective injector 186. Each calibration unit 270 can comprise a vial 271 including indicia configured to denote the level of medicament within the vial. In use, the injector can administer a dose to the vial 271 and a user can use the indicia to determine the actual volume of the dose. Alternatively, an automated unit, such as control unit 126, can determine the actual volume of the dose.

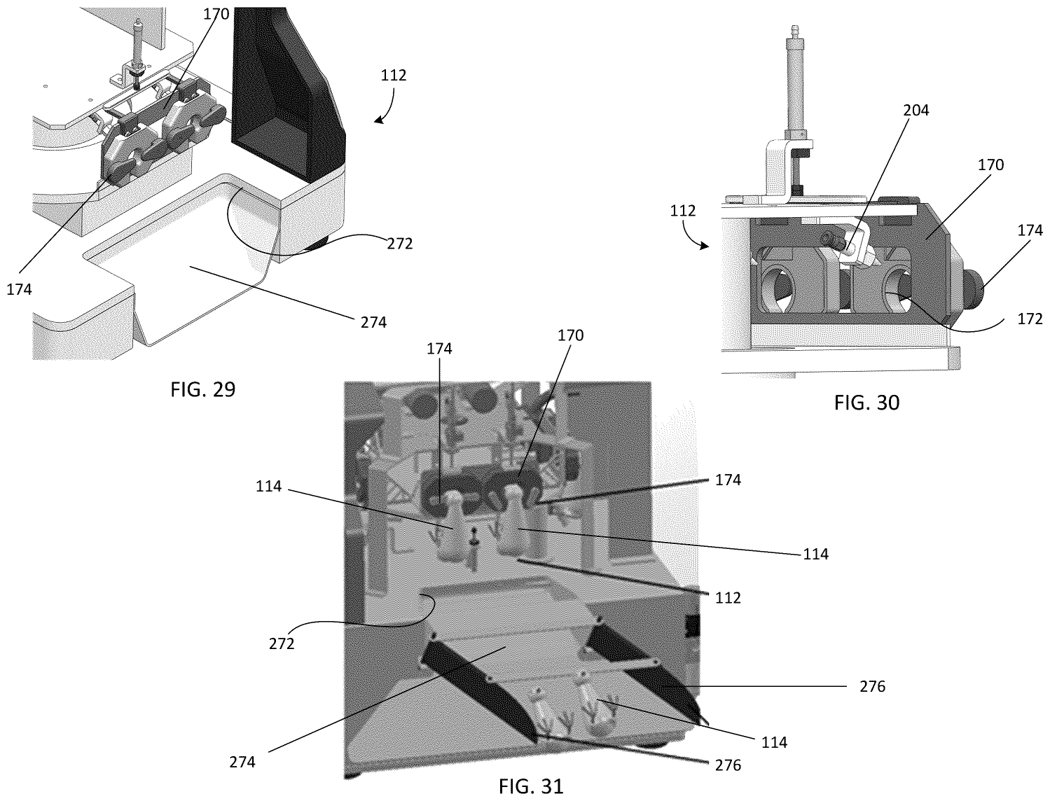

Referring now to FIGS. 29-31, once the aerosolized medicament has been administered to the subject 114 at the spraying station 110, the administration device 100 can automatically rotate the holder 170 to the release station 112. Once the holder 170 is positioned at the release station 112, the clamps 174 can be released, releasing the subject's head and thereby allowing the subject 114 to exit the administration device 100. The one or more base supports 176 can also be released, for example, by pivoting inwardly toward the central axis of the administration device 100. In some embodiments, the release station can comprise one or more air sprayers configured to spray, for example, a puff of air at the subjects 114 to cause the subject to retract their head from the holder 170 once the clamps are released.

As shown in FIG. 29, the release station 112 can comprise a cutout 272 and a sloped ramp portion 274. The ramp portion 274 can be configured to allow the subject(s) 114 to slide down the ramp 274 and out of the administration device 100, for example, into a container.

In some embodiments, the ramp 274 can comprise one or more ramp guides 276 configured to guide the subjects 114 into, for example, a container on a conveyor belt. The guides 276 can be pivotably coupled to the administration device 100. Once a selected container has been filled with subjects 114, the guides 276 can pivot to direct the subjects into a second container, reset the container counter, etc.

In some embodiments, the administration device 100 can be cleaned and/or disinfected by, for example, replacing the one or more medicament containers 138 with one or more containers of cleaning fluid (e.g., detergent, water, alcohol, oil, etc.). The cleaning fluid can travel through the tubing and pumps of the administration device 100 semi-automatically. For example, the administration device 100 can comprise a button that activates a cleaning function of the device and/or the cleaning function can be remotely controlled using Bluetooth, Wi-Fi, or other communication RF or IR system, managed using a mobile app or other remote control system. When in the cleaning function the pumps 190 can move cleaning fluid through the system. Then the cleaning fluid can be replaced with water and/or oil, which is flowed through the system using the pumps 190. The cleaning function can have several steps including several different fluids, for example, detergent, water, alcohol, and/or oil. The control unit 126 can be configured to provide one or more alerts when the administration device 100 needs maintenance, for example, alerting a user when the device is in need of cleaning.

As mentioned previously, in some embodiments, the control unit 126 can be configured to receive data from and/or transmit data to a remote device. In such embodiments, the remote device can be configured to store data from, transmit data to, and/or remotely control the administration device 100. The remote device can be, for example, a general purpose computer, a hand-held mobile device (e.g., a cell phone or tablet), and/or any type of accessory therefore (e.g., a “smart watch” etc.).

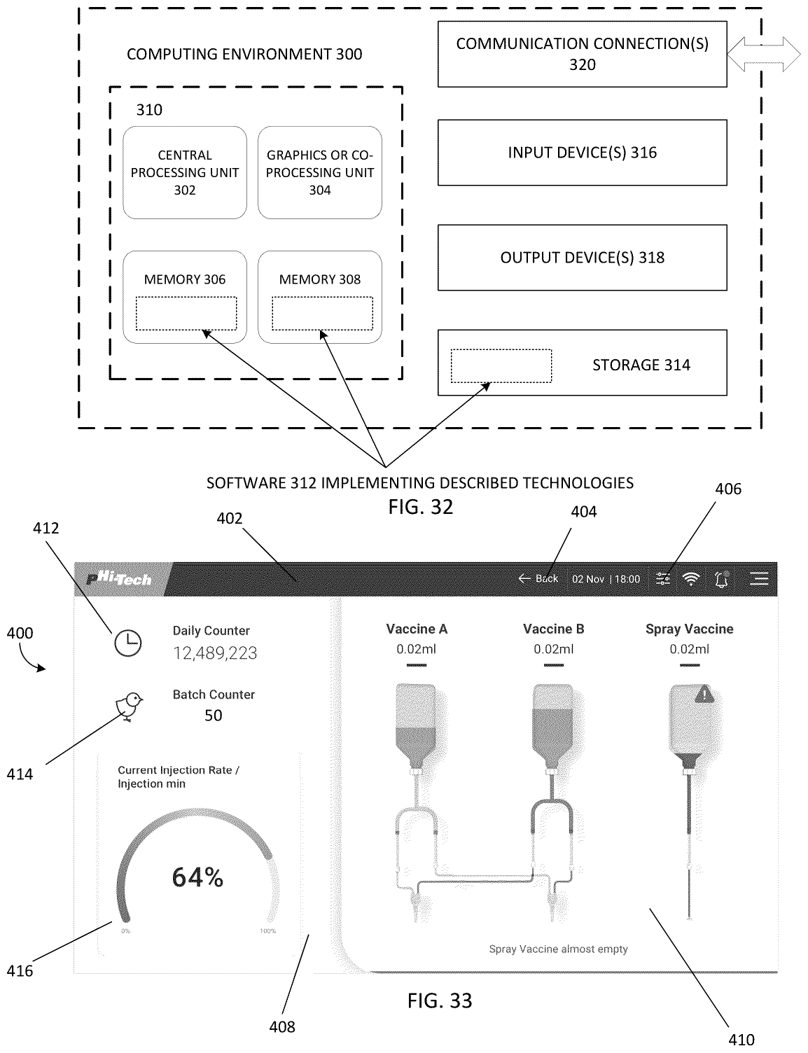

The following is a general description of a computing environment suitable for use with the disclosed control unit 126. FIG. 32 depicts a generalized example of a suitable computing environment 300 in which software and control algorithms for the described innovations may be implemented. The computing environment 300 is not intended to suggest any limitation as to scope of use or functionality, as the innovations may be implemented in diverse general-purpose or special-purpose computing systems. For example, the computing environment 300 can be any of a variety of computing devices (e.g., desktop computer, laptop computer, server computer, tablet computer, gaming system, mobile device, programmable automation controller, etc.).

With reference to FIG. 32, the computing environment 300 includes one or more processing units 302, 304 and memory 306, 308 (e.g., for storing sequence data and/or system input data). In FIG. 32, this basic configuration 310 is included within a dashed line. The processing units 302, 304 execute computer executable instructions. A processing unit can be a general-purpose central processing unit (CPU), a processor in an application-specific integrated circuit (ASIC), or any other type of processor. In a multi-processing system, multiple processing units execute computer-executable instructions to increase processing power. For example, FIG. 32 shows a central processing unit 302 as well as a graphics processing unit 304. The tangible memory 306, 308 can be volatile memory (e.g., registers, cache, RAM), non-volatile memory (e.g., ROM, EEPROM, flash memory, etc.) or some combination of the two, accessible by the processing unit(s). The memory 306, 308 stores software 312 implementing one or more innovations described herein, in the form of computer-executable instructions suitable for execution by the processing unit(s).

A computing system may have additional features. For example, in some embodiments, the computing environment 300 includes storage 314, one or more input devices 316, one or more output devices 318, and one or more communication connections 320. An interconnection mechanism (not shown) such as a bus, controller, or network, interconnects the components of the computing environment 300. Typically, operating system software (not shown) provides an operating environment for other software executing in the computing environment 300, and coordinates activities of the components of the computing environment 300. In some embodiments, the computing system can include virtual network computing (VNC) functionality configured to allow operators to access the control unit 126 and computing environment 300 from a remote location. For example, the computing environment 300 can have remote dial-in capability. The VNC functionality can allow an operator to remotely access the computing environment in order to, for example, perform maintenance or live monitoring of the administration device 100, to send configuration parameters to the system, to set and/or reset disposable replacements, and/or to train an operator on the use of the administration device 100.

The tangible storage 314 may be removable or non-removable, and includes magnetic disks, magnetic tapes or cassettes, CD-ROMs, DVDs, or any other medium that can be used to store information in a non-transitory way and can be accessed within the computing environment 300. The storage 314 stores instructions for the software 312 implementing one or more innovations described herein (e.g., for storing sequence data, temperature data, template type data, location, date, etc.). In some embodiments, the storage can be a “cloud-based” system configured to store data, allow access to data, and/or generate reports. For example, data logs can be sent to a cloud system and reports can be generated therefrom. Users (including, for example, clients) can access the cloud system remotely through using selected log-in credentials.

The input device(s) 316 can be, for example: a touch input device, such as a touchscreen display, keyboard, mouse, pen, or trackball; a voice input device; a scanning device (e.g., a barcode scanner, RFID scanner, NFC scanner); any of various sensors (e.g., the quantity indicator, speed indicator, location unit, etc.); another device that provides input to the computing environment; or combinations thereof. The input device(s) can be remote from the control unit. The output device(s) 318 can be a display, printer, speaker, CD-writer, transmitter, or another device that provides output from the computing environment 300.

The communication connection(s) 320 enable communication over a communication medium to another computing entity. For example, the communication connection(s) can enable communication between the control unit 126 and a remote input device, for example, a phone app, or a computer browser. The communication medium conveys information, such as computer-executable instructions or other data in a modulated data signal. A modulated data signal is a signal that has one or more of its characteristics set or changed in such a manner as to encode information in the signal. By way of example, and not limitation, communication media can use an electrical, optical, RF, Wi-Fi, Bluetooth, or other carrier.

Any of the disclosed methods can be implemented as computer-executable instructions stored on one or more computer-readable storage media (e.g., one or more optical media discs, volatile memory components (such as DRAM or SRAM), or nonvolatile memory components (such as flash memory or hard drives)) and executed on a computer (e.g., any commercially available computer, including smart phones, other mobile devices that include computing hardware, or programmable automation controllers). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C, C++, Java, Perl, JavaScript, Adobe Flash, or any other suitable programming language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communications means include, for example, the Internet, an intranet, software applications, cable (including fiber optic cable), magnetic communications, electromagnetic communications (including RF, microwave, and infrared communications), electronic communications, or other such communication means.

As mentioned previously, the remote device can include an application, or “app,” configured to control the administration process and/or track information relating to the administration process. In some embodiments, the control unit 126 can transmit real-time information to the remote device which can be displayed by the app.

The display can be configured to display a graphical user interface (GUI) comprising one or more data outputs (e.g., a daily counter, a batch counter, a current injection rate, a medicament volume tracker, medicament temperature, an alarm/alert, etc.) from the administration device 100. In some embodiments, the display can be a touchscreen display/UI and is configured to accept user input(s). The display can have any configuration suitable to display one or more of: (1) system input information such as medicament type; (2) system output information such as a daily counter, a batch counter, a current injection rate; (3) instructions to a user; (4) alerts/alarms; or (5) any combination thereof. In some embodiments, the display can be configured such that a user can input data to the control unit 126 via the display.

FIG. 33 shows an exemplary embodiment of a graphical user interface (GUI) 400, that includes display areas for displaying process input and output parameters. In the illustrated example, the GUI 400 can include a navigation pane 402 (which can include, e.g., one or more navigation buttons, such as button 404, and buttons 406 accessing one or more settings) and a display pane 408. A user can use the navigation pane 402 to navigate between displays by clicking and/or pressing the buttons 404, 406. The display pane 408 can comprise, for example, a medicament tracking pane 410, a daily counter pane 412, a batch counter pane 414, and a batch tracking pane 416. In some embodiments, clicking and/or pressing on each pane can enlarge the pane (e.g., like a pop-up menu) and/or provide additional information about the pane.

The medicament tracking pane 410 can be configured to display indicators representing each medicament container 138 and displaying the volume of medicament within each container 138. The daily counter pane 412 can be configured to display the number of subjects to whom medicament has been administered on a selected day. The batch counter pane 414 can be configured to display the number of subjects in a first container disposed adjacent the release station. For example, the container can be full when 50 subjects are disposed within the container. Once the batch counter reaches 50, the control unit 126 can actuate the administration device 100 such that the subjects are disposed in a second container. The batch counter can then restart from zero. The injection rate tracking pane 416 can display the rate of injections per minute (e.g., the injection speed of the device).

In certain embodiments, the control unit 126 of the administration device 100 can further comprise a detection system, which can be configured to identify one or more malfunctions and/or other issues in the system. Some malfunctions may prevent accurate injection/spraying of a medicament to a subject, prolong the duration of each injection/spraying, result in incomplete injection/spraying, and/or result in improper dosing. In some embodiments, the detection system can further be configured to send an alert and/or alarm to the user via the control unit 126 identifying the malfunction and/or other issue. In some embodiments, the control unit 126 can transfer the one or more alerts and/or alarms directly to a cloud-based system, such alerts/alarms can then be sent to one or more remote devices and/or logged in the cloud-based system for review at a later time. In some embodiments, the alerts and/or alarms can be sent to one or more remote devices using an SMS generator such that the messages are received by the remote devices as SMS messages.

Such a detection system can comprise various mechanisms, for example, a probe at the needle or needle head that identifies an early withdrawal of the needle 188 prior to the completion of the injection, a probe coupled to the dosing chamber 218 that identifies whether the piston 220 head moves all the way to the end of the dosing chamber 218, a probe at the piston 220 head that identifies whether the head of the piston 220 moves backwardly before the piston head moves all the way to the end of the dosing chamber (e.g., indicative of incomplete administration), a sensor that identifies whether the piston 220 does not move or moves very slowly (e.g. using the encoder and an internal clock), which can be indicative of a clog, and/or a sensor that identifies whether the piston 220 moves too fast (e.g. using the encoder and an internal clock), which can be indicative of an air bubble or leakage, a sensor that identifies whether any subjects 114 are present in the holder 170, a sensor that determines whether the subjects 114 are correctly positioned within the holder 170, a sensor that determines whether a user's hands are present in the loading station, and/or any combination thereof.

The detection system can further comprise one or more temperature sensors configured to determine the temperature of the medicament and send an alert and/or alarm if the temperature is too high or too low. The detection system can further comprise one or more pressure gauges. The pressure gauges can be used to determine whether there are blockages and/or air bubbles in the administration device 100, whether the medicament container(s) 138 are empty, and/or whether the needle 188 is blocked, among other uses. The detection system can further determine when the administration device 100 is in need of cleaning and/or disinfection, for example, by measuring the time lapse and usage of the device 100 since the last cleaning process.

The detection system can further function to calculate the remaining amount of medicament in each container 138 (e.g., by multiplying the number of doses by the injection and/or spray dosage), measure the current used to activate the motor(s) 260, which can correspond to the presence of an air bubble, leakage or a clog, etc. The detection system can further be configured to measure the duration of each dose and/or the electric current used, wherein any additional duration and/or current used beyond a certain amount can indicate clogging within the system, and any decrease in duration and/or current used beyond a certain amount can indicate an air bubble or leak within the system, or the end of the medicament in the container.

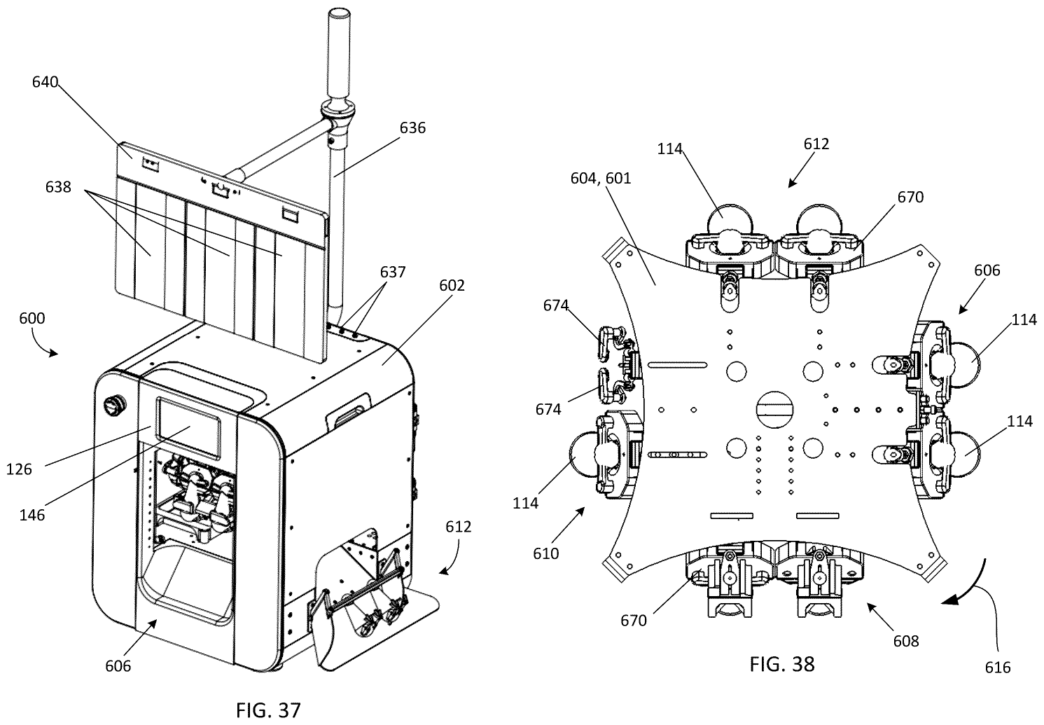

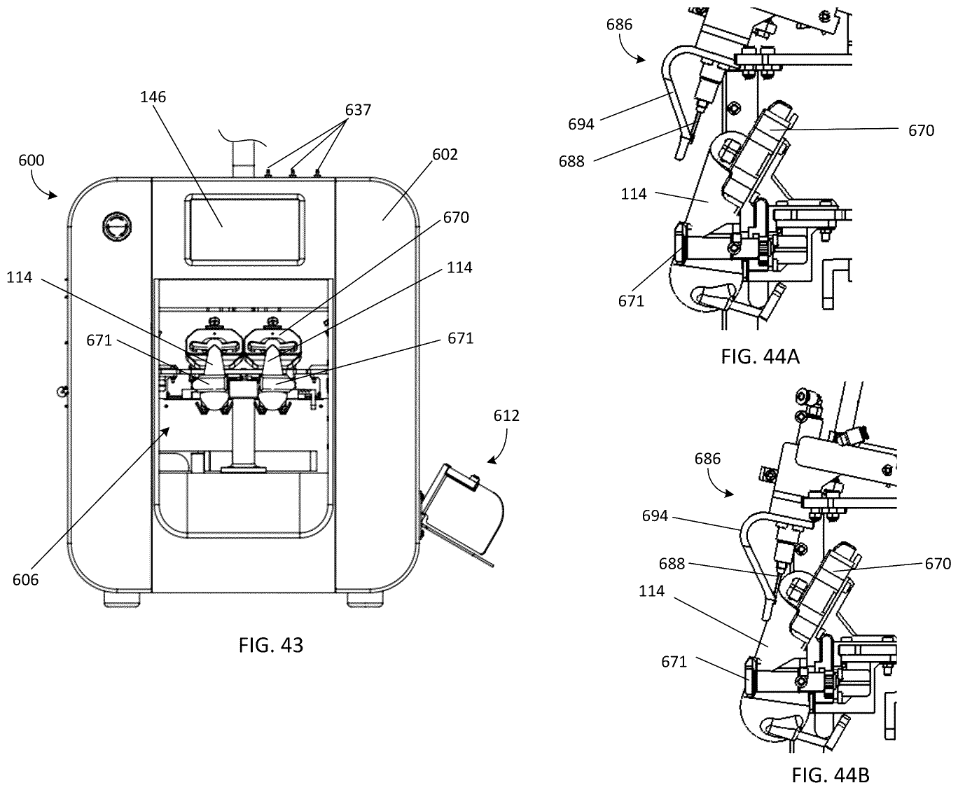

FIGS. 37-54 illustrate another exemplary embodiment of an administration device 600. Administration device 600 is similar to or the same as administration device 100 except where specifically described otherwise. Any of the embodiments described above with respect to administration device 100 can also apply to administration device 600 unless specifically stated otherwise.

Referring to FIG. 37, administration system 600 generally comprises a housing 602, a carousel 604 (see FIG. 38), and a plurality of stations each configured for a selected function. In the illustrated embodiment, as shown in FIG. 38, the administration device 600 comprises four stations: a loading station 606, an injection station 608, a spray station 610, and a release station 612. Subjects 114 can be loaded into the carousel 604 (e.g., manually or automatically) at the loading station 606. Once loaded, the carousel 604 rotates the subjects 114 through each station (e.g., in the direction of arrow 616) before automatically releasing the subjects at the releasing station 612.

The administration device 600 can be used to administer one or more medicaments to a subject. For example, in the embodiment shown in FIGS. 37-54, each subject can be administered two injected medicaments (e.g., via the injection station 608) and one spray medicament (e.g., via the spray station 610). The administration device 600 can be configured to administer medicament to between 5000 and 7000 subjects per hour.

Similar to administration device 100, administration device 600 can comprise a control unit (e.g., control unit 126 comprising display 146 described previously). The control unit 126 can be used to control the medicament administration process and/or track data related to the administration process.

The administration device 600 can comprise one or more supports 636 configured to support one or more medicament containers or medicament container holders 638. The support 636 can comprise hanger portion 640 configured to hold the one or more medicament containers 638. Each medicament container 638 can hold, for example, between 200 ml and 2 L of medicament. In some embodiments, the medicament containers can be bags, in other embodiments, they can be solid containers. In some embodiments, each medicament container 638 can comprise a sensor configured to detect whether a container is present, the volume level within a container, and/or whether the container is empty.

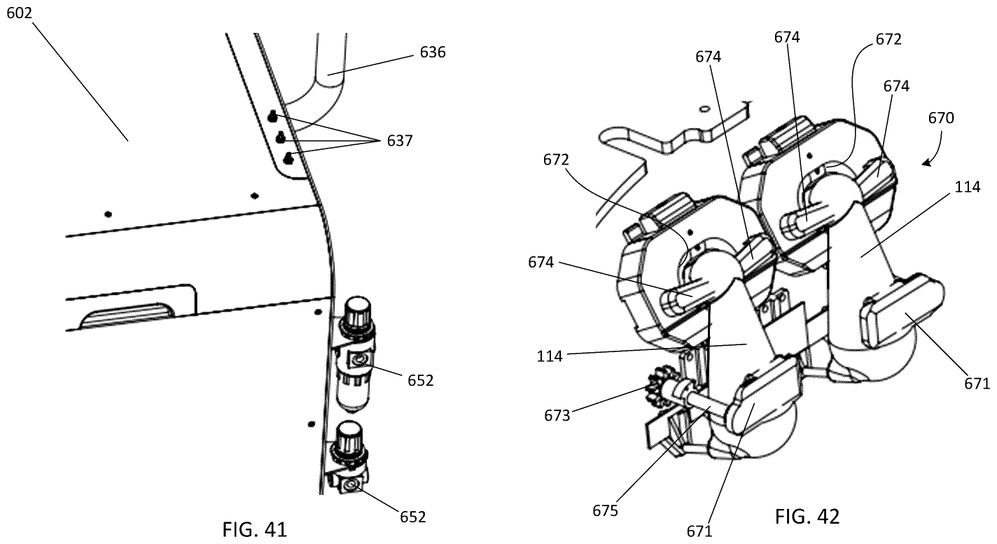

The medicament containers 638 can be coupled to the administration device 600 via one or more connecting tubes (such as connecting tubes 144 described previously) coupled to inlets 637 extending from an outer surface of the housing 602. As shown in FIG. 41, in the illustrated embodiment the administration device has three inlets 637, each corresponding to a respective medicament container 638. However, in other embodiments, the administration device can have a greater of fewer number of inlets.

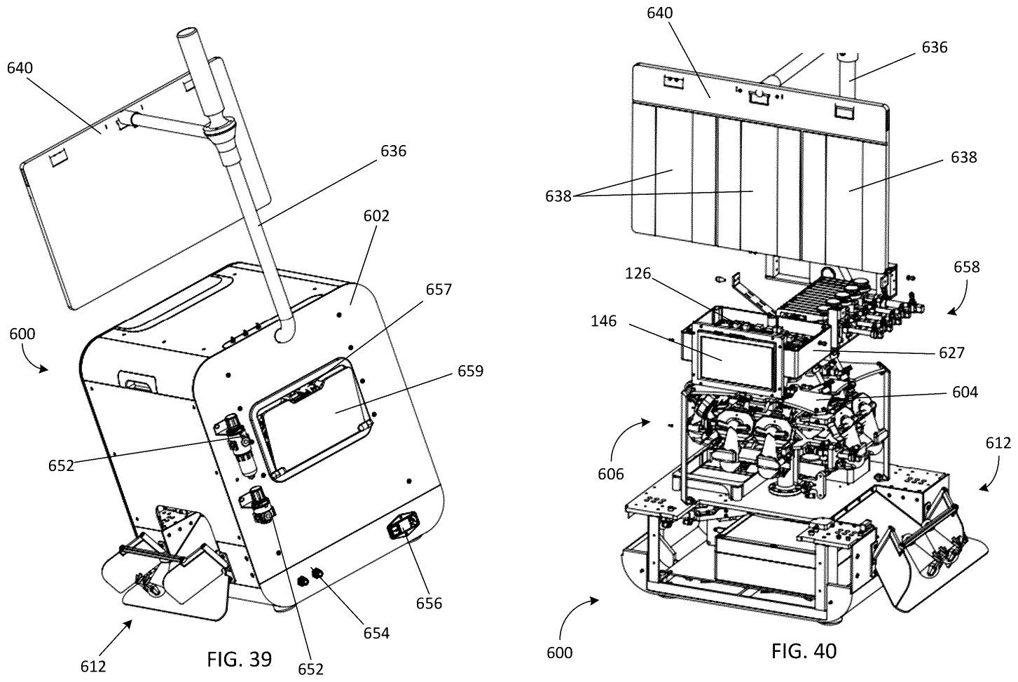

Referring to FIG. 39, the administration device 600 can comprise one or more inlet valves 652 (e.g., for pneumatic air), a power inlet 654, and a power switch 656. In the embodiment illustrated in FIG. 39, the administration device comprises two inlet valves 652, each of which comprises a pressure gauge. The first pressure gauge can determine the main pressure of the administration device (e.g., 6 Bar in some particular embodiments), and the second pressure gauge can determine the injection pressure (e.g., 2 Bar in some particular embodiments). The inlet valves 652 are configured to allow the device 600 to be coupled to one or more pneumatic sources. The power inlet is configured to allow the device to be coupled to a power source (e.g., an electrical outlet, batteries, a solar panel, USB, etc.), and the power switch 656 is configured to turn the device on or off.

In some embodiments, each station can comprise a window or opening 657 (see e.g., opening 657 in FIG. 39), configured to allow a user to reach into the administration device 600 to, for example, remove one or more subjects 114, clean the device, and/or replace one or more components of the device without needing to disassemble the entire device. The openings 657 can comprise a door 659 configured to block access to the opening unless the door is open. The openings 657 can comprise a sensor such that the device 600 will not operate if the door 659 is open and/or such that the device 600 will automatically stop operating if the door 659 is opened during use of the device.

Referring to FIG. 40, the administration device 600 (shown without housing 602) can further comprise a dosing system 658 coupled to the injection station 608 and/or spray station 610. As mentioned, the device 600 can comprise a control unit (e.g., control unit 126 mentioned previously). The control unit 126 can comprise a PCB and/or other electronics components housed within a box 627 adjacent the dosing system 658.

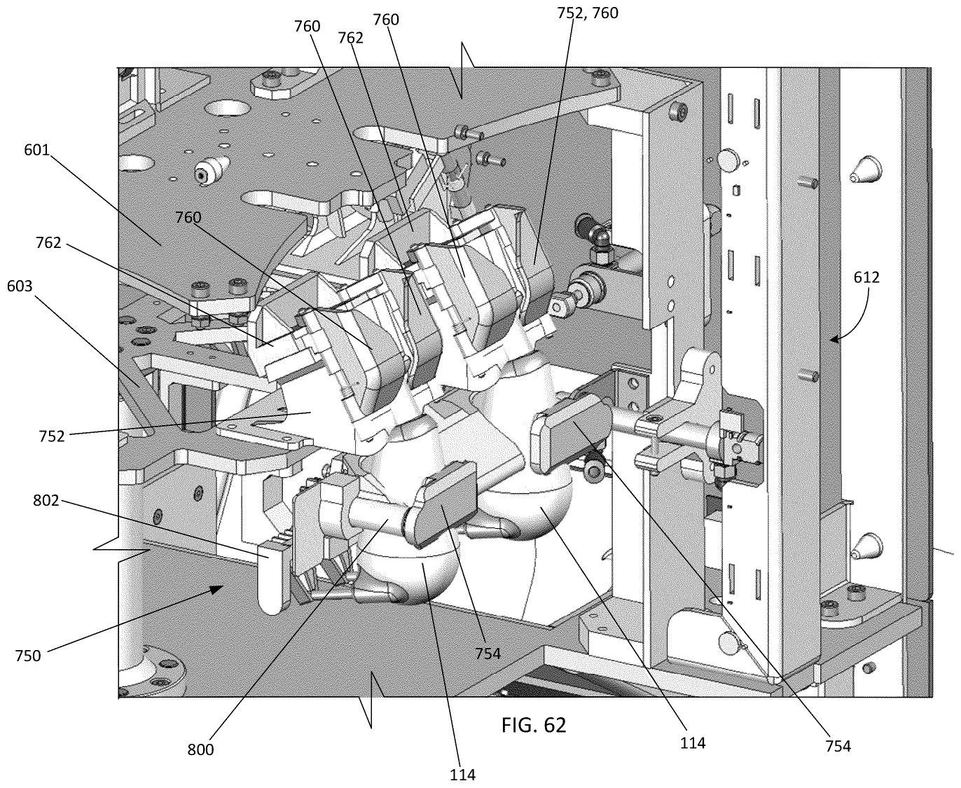

Referring to FIG. 42, the carousel 604 can comprise one or more holders 670 for holding one or more subjects 114. In the illustrated embodiment, each holder 670 is configured to retain or hold two subjects 114. However, in other embodiments, each holder can be configured to retain any number of subjects, for example, one subject, three subjects, four subjects, etc. The holders 670 can be coupled to the carousel 604 in a rotatable manner such that the holders 670 can rotate relative to the plurality of stations (e.g., stations 606, 608, 610, and 612). For example, the holders 670 can be coupled to a movable portion 603 (FIG. 62) of the carousel 604 and the stations can be coupled to a stationary portion 601 (FIG. 62) of the carousel.

Each holder 670 can comprise one or more openings 672 into which the subject's head can extend, a pair of clamps 674 configured to restrain the subject 114 against movement relative to the holder 670, and a back support 671. The clamps 674 can have an open position and a closed position (see FIG. 42). During loading of the subject(s) 114 at the loading station 606, a user can insert the subject's head into the opening 672 and the clamps 674 will automatically actuate to restrain the subject 114. In other embodiments, the clamps 674 can be actuated manually (e.g., by pressing down on the clamps with, for example, a finger). In still other embodiments, a user can insert the subject's head into the opening 672 and actuate the clamps 674 by, for example, pressing a button on the control unit 126. In additional embodiments, the button can be located such that a user can press the button with a foot.

Once the clamps 674 are in the closed position, each back support 671 can rotate into a closed position such that it engages the subject's back to further restrain the subject against movement relative to the holder 670. As shown in FIG. 42, the back support 671 can be coupled to a toothed component or gear 673 via an arm 675 such that the back support 671 can rotate between an open position and a closed position (e.g., as shown in FIG. 42). In other embodiments, the back supports 671 can be hingedly coupled to the holder 670.

Once the subjects 114 have been loaded into the holder 670 via the loading station 606, the movable portion 603 of the carousel 604 can rotate, moving the subjects from the loading station 606 to the injection station 608. In some embodiments, the loading station 606 can comprise a sensor configured to determine whether one or more subjects 114 have been loaded into the holder 670. In some embodiments, once at least one subject has been loaded, the administration device 600 will automatically rotate the holder 670 to the next station, for example, the injection station 608. In some embodiments, the loading station can comprise one or more sensors configured to determine whether the user's hands are within the loading station. The administration device 600 will not automatically rotate until the user's hands have been removed from the loading station 606.

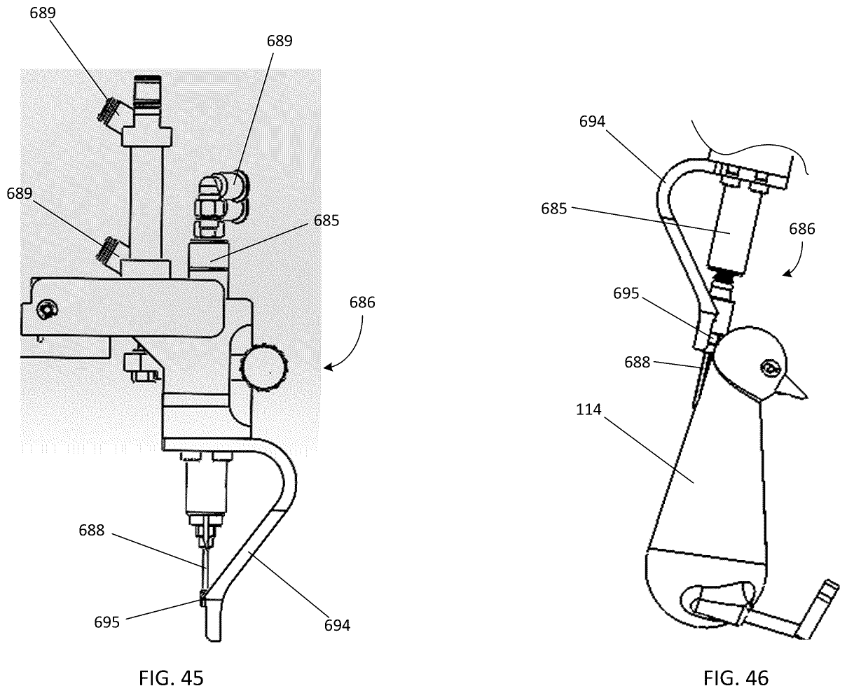

Referring to FIGS. 44A-47, the injection station 608 can comprise one or more injectors 686 similar to injectors 186 described previously. The injectors 686 are configured to subcutaneously administer a selected dosage of medicament to a subject 114. As shown in FIG. 45, each injector 686 can comprise a needle 688 fluidly coupled to one or more pumps 190 (see e.g., FIG. 20). The pumps 190 can be located, for example, in the dosing system 658. The needle 688 can be coupled to the one or more pumps via, for example, one or more flexible tubes. The flexible tubes can be coupled to the injector 686, for example, via inlets 689. The needle 688 can be removably coupled to the injector 686 (e.g., using a luer lock), such that the needle 688 can be easily removed and/or replaced without disassembling the injector 686 and/or the administration device 600. The injector 686 can comprise one or more additional inlets 689 coupled to one or more pneumatic actuators and configured to allow the needle 688 to be pneumatically actuated such that it can move relative to the injector.

The injector 686 can be pivotably coupled to the carousel 104 such that the injector 686 movable between a ready position (see e.g., FIG. 44B) and a non-ready position (scc e.g., FIG. 44A). When the injector is in the ready position, if a subject 114 is present, the injector can rest against the subject. In some embodiments, once the needle 688 has reached the selected injection depth, one or more doses of medicament can be administered automatically to the subject. As shown in FIG. 44A, when in the non-ready position, if a subject 114 is present, a distal edge of the needle 688 will not contact the subject.

Once the injector is in the ready position and the needle 688 is positioned against the subject 114, the needle 688 can be actuated such that it moves relative to the injector 686. For example, the needle can be coupled to a pneumatic piston 685 configured to actuate the needle. Accordingly, the needle can be movable between a retracted position and an extended position wherein the distal edge of the needle 688 pierces the subject.

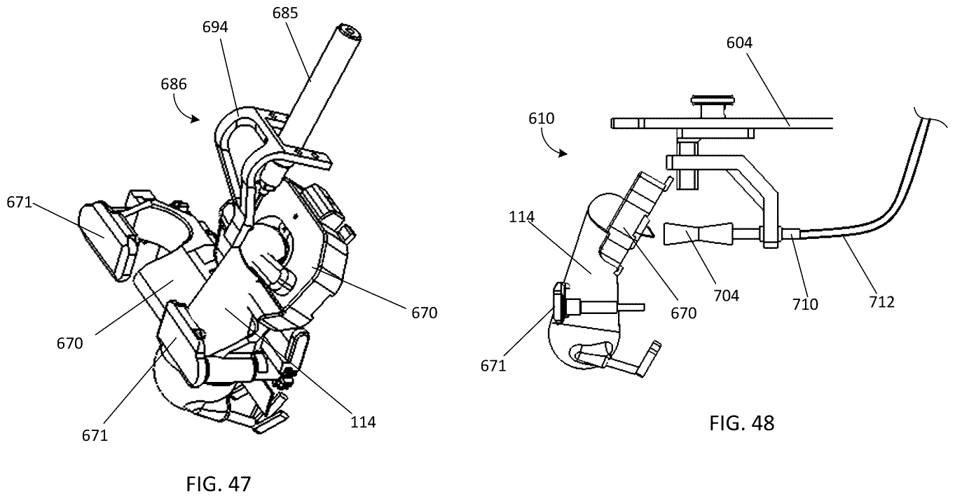

Each injector 686 can further comprise a respective guide 694. As best seen in FIG. 47, the needle guide can be an elongated member having a curved Y-shape. As shown in FIG. 45, the guide 694 can comprise a projection 695 extending from an inner surface of the guide. The projection 695 can comprise an inner channel or bore through which the needle 688 can extend when in the extended position. Accordingly, when the injector 686 is in the ready position, the projection 695 can be positioned against the subject 114 to guide the needle 688 into the subject at a selected angle. For example, the projection 695 can be used to position the needle 688 for subcutaneous injections. Subcutaneous injections require that the needle penetrate the subject's skin but stop prior to penetration of the subject's muscle such that the medicament is deposited between the skin and the muscle. Such injections require that the needle 688 enter substantially parallel to the skin (see e.g., FIG. 46). As used herein, an object is “substantially parallel” with respect to a reference object or plane when the object is oriented at an angle of +20° or less with respect to the reference object or plane.

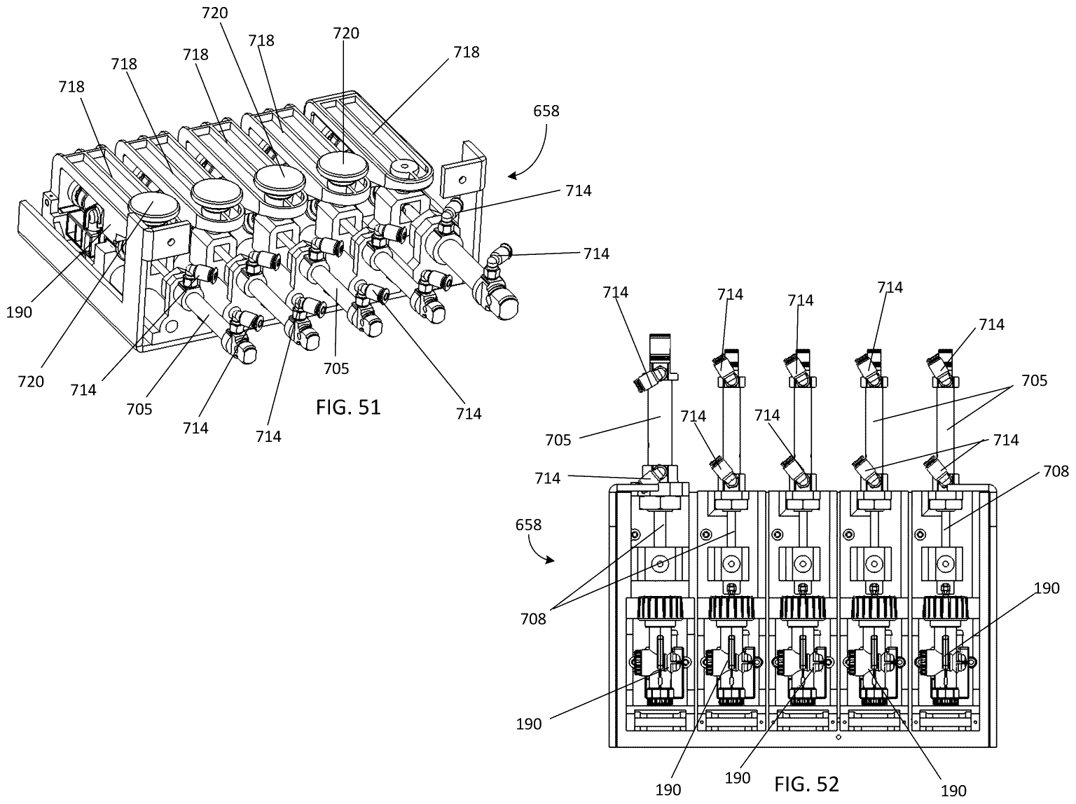

Once the needle 688 has reached a selected depth within the subject (e.g., as determined by a sensor attached to the pneumatic piston and/or the needle), a pump 190 of the dosing system 658 (see e.g., FIG. 51) can actuate to automatically inject a dose of medicament. In embodiments wherein the needle 688 is coupled to two or more pumps 190, each dose of medicament can be injected simultaneously. In other embodiments, two or more doses of medicament can be injected sequentially. Once the subjects 114 have received the dosc(s) of medicament, the administration device 600 can rotate the holder 670 to the next station, for example, the spraying station.

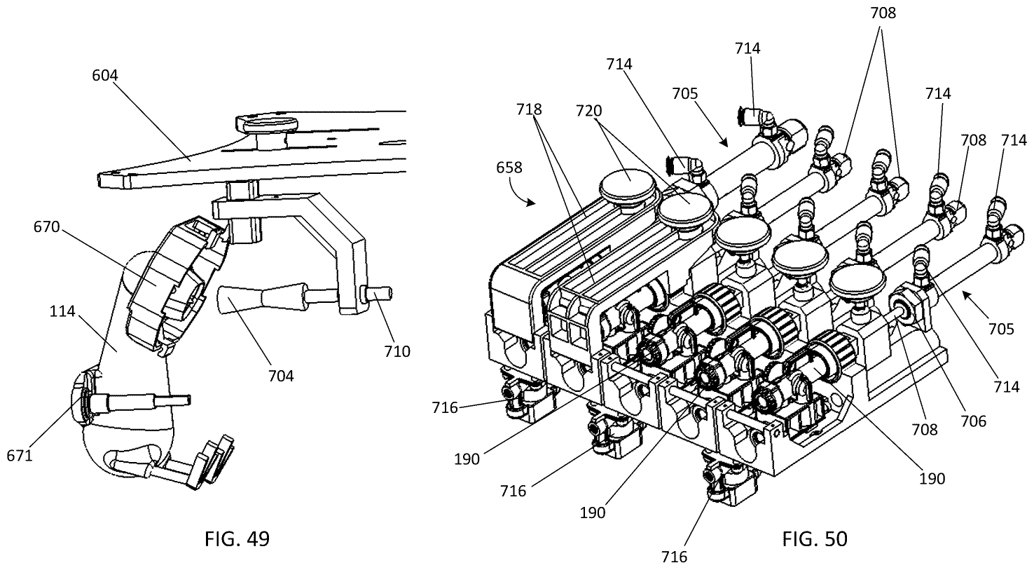

Referring to FIGS. 48-49, the spraying station 610 can be similar to spraying station 110 described previously, and can comprise one or more spray applicators or nozzles 704. Each spray nozzle 704 can be configured to administer a medicament in the form of a plurality of droplets to one or more mucosal tissues (e.g., the eyes, nose, and/or mouth) of the subject 114. As discussed previously with respect to spraying station 110, certain vaccinations with inactive/dead viruses can be ineffective as injected vaccines but too pathogenic to inject directly. Accordingly, such live vaccines can be administered via spraying to the mucosa of the subject. In some particular embodiments, the spraying station 610 can be configured to administer 0.1 ml of medicament per subject.

As shown in FIG. 48, each spray nozzle 704 can comprise a connector 710 configured to couple one or more flexible tubes 712. The flexible tubes can couple the spray nozzle to one or more pumps 190 (see e.g., FIG. 20). The pumps 190 can be located, for example, in the dosing system 658.

In some embodiments, as discussed previously with respect to spraying station 110, the spraying station 610 can comprise a mechanism for controlling and/or adjusting the size of the spray droplets, and a funnel or hood to guide the sprayed medicament to the mucosal tissue of the subject. In some embodiments, the spraying station 610 can further comprise a fan such as a fan 212 described previously.

Once the aerosolized medicament has been administered to the subject 114, the administration device 600 can automatically rotate the holder 670 to the next station, for example, the release station 612.

Referring now to FIGS. 50-53, as mentioned previously, each injector 686 and/or spray nozzle 704 can be coupled via one or more flexible tubes (e.g., tubes 712) to one or more pumps 190. The one or more pumps 190 and associated tubing can form a dosing system 658 which can be coupled to the one or more medicament containers 638 and through which the medicament can flow. In the embodiment illustrated in FIGS. 50-53, the dosing system 658 can comprise five pumps 190. One or more pumps can be coupled to the injectors 686, and one or more pumps can be coupled to the spray nozzles 704. For example, each needle 688 can be coupled to two pumps 190 via one or more tubes, which allows administration of the different medicaments by different pumps 190 to the same needle 188, the remaining pump can be coupled to one or more spray nozzles 704 of the spray nozzle station 610. In other embodiments, the dosing system 658 can comprise any number of pumps 190 arranged in any configuration. For example, in some embodiments, each needle 688 is coupled to a single pump 190. In other embodiments, each spray nozzle 704 can be coupled to two or more pumps 190, or each spray nozzle 704 can be coupled to a single pump 190.

As mentioned previously and as shown in FIG. 20, each pump 190 can be a piston or plunger pump comprising a dosing chamber 218, a piston 220, and one or more one-way valves (e.g., valves 222 and 224). When actuated, pump 190 can pull medicament into the dosing chamber 218 through the first or inlet valve 222, as shown by arrow 225, and can then push the medicament from the dosing chamber 218 to the injector 686 and/or one or more spray nozzles 704 through the second or outlet valve 224.

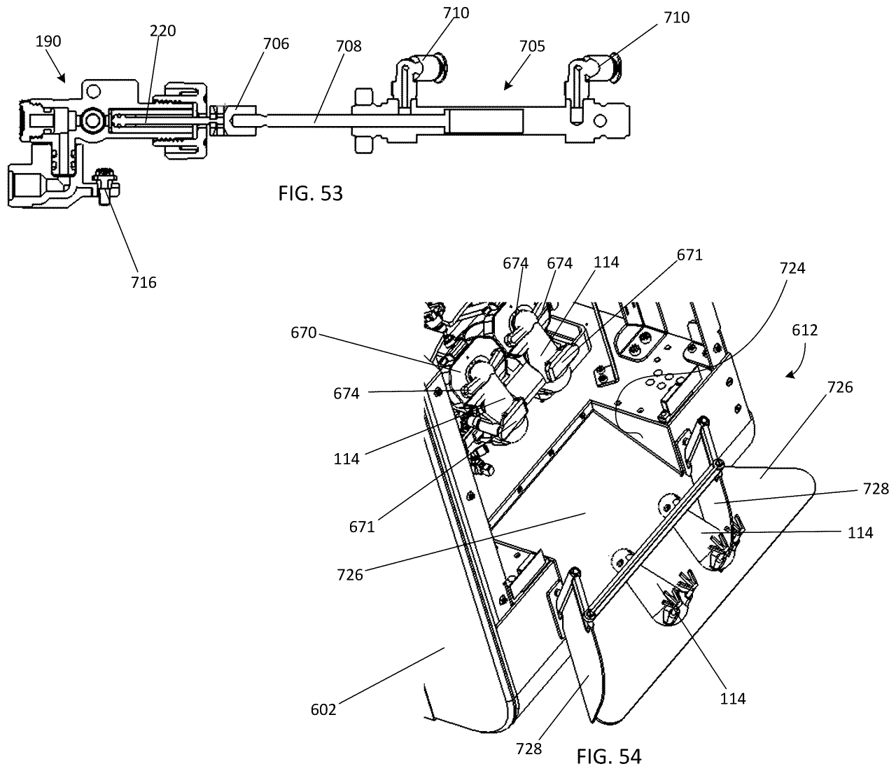

Referring to FIG. 50, the piston 220 (see FIG. 53) of each pump 190 can be coupled to a pneumatic actuator 705. Each pneumatic actuator 705 can comprise an adapter 706, a pneumatic piston 708 (see FIG. 53), and one or more air inlets 714. As shown in FIG. 53, the adapter 706 can be configured to couple the pneumatic piston 708 to the pump piston 220 such that movement of the pneumatic piston 708 causes corresponding movement of the pump piston 220. Each air inlet 714 can be coupled (via, for example, a tube or hose) to a pneumatic source configured to actuate the pneumatic piston 708.

In use, the pneumatic piston 708 can be actuated to move the piston 220 out of the dosing chamber, causing the pressure in the dosing chamber 218 (see FIG. 20) to be reduced. Medicament can then flow into the dosing chamber 218 through the first one-way valve. Once the dosing chamber is filled, the pneumatic piston 708 can be actuated to move the piston 220 into the dosing chamber, causing the medicament to flow through the second one-way valve. The positive pressure created by the piston 220 pushes the medicament through a connecting tube through the needle and/or spray nozzle of the administration device, and into the subject.

The dosing system 658 can further comprise one or more pressure sensors 716. For example, in the illustrated embodiment shown in FIGS. 50-53, the dosing system 658 can comprise three pressure sensors 716. Two pressure sensors 716 can each be coupled to two pumps 190 (e.g., the pumps providing medicament to the injection station), and the remaining pressure sensor can be coupled to one pump 190 (e.g., the pump providing medicament to the spraying station). The pressure sensors 716 can be configured to detect whether there is sufficient pressure in the line (e.g., detect the presence of air in the line). If the sensors detect air, the control unit 126 can provide an alert of such to the operator. This can alert the operator that, for example, there is insufficient medicament in the medicament containers and they need to be replaced, or some other issue has occurred allowing air to enter the system.

The pressure sensors 716 can further be configured to determine whether and when the administration device 600 has been sufficiently primed and is ready for use. For example, during the priming process (e.g., initially coupling the medicament to the dosing system and preparing the system for use) there may be air in the system, once the pressure sensors detect that the air is gone, the control unit can alert the user that the system is ready for usc.

In some embodiments, in lieu of or in addition to a dosage adjustment system (e.g., dosage adjustment system 160 described previously), the dosages of medicament for the injectors and/or spray nozzles can be selected using a modular pump system. In such embodiments, the administration device 600 can comprise various fixed-dosage pumps 190 having dosing chambers 218 of various sizes. Depending on the selected dosage of medicament, the user can select the pump having the dosing chamber of the corresponding size. The user can then couple the pump to the dosing system 658. In such embodiments, the pumps can comprise external indicia (e.g., color coding, symbols, lettering, etc.) alerting the user to the size of the dosing chamber. In some embodiments, the control unit 126 can include a library of various vaccine types and selected dosages. The user can select one or more vaccine(s) and dosage(s), and the control unit 126 can determine which pump unit(s) the user should insert into the dosage adjustment system.

Referring to FIG. 50, in some embodiments, each pump 190 can comprise a cover 718 and a knob 720. Each cover 718 can be configured to cover a respective pump 190 and retain the pump in place and/or prevent the pump from being inadvertently moved or removed. The knobs 720 can be configured to hold the covers 718 in place, as shown in FIG. 51.

Referring to FIG. 54, once the aerosolized medicament has be administered to the subject 114 at the spraying station 610, the administration device 600 can automatically rotate the holder 670 to the release station 612. Once the holder 670 is positioned at the release station 612, the back support 671 can be disengaged (e.g., by pivoting upwardly) and the clamps 674 can be released, releasing the subject's head and thereby allowing the subject 114 to exit the administration device 600.

As shown in FIG. 54, the release station 612 can comprise a cutout 724 and a sloped ramp portion 726. The ramp portion 726 can be configured to allow the subject(s) 114 to slide down the ramp 726 and out of the administration device 600, for example, into a container. The ramp 726 can comprise one or more ramp guides 728 configured to guide the subjects 114 into, for example, a container. The guides 728 can be pivotably coupled to the administration device 600. Once a selected container has been filled with subjects 114, the guides 276 can pivot to direct the subjects into a second container, reset the container counter, etc. In some embodiments, once a selected number of subjects (e.g., 50-10) have be deposited in a first selected container, the control unit 126 can automatically actuate the guides 728 pivoting them such that they direct the subjects into a second selected container.

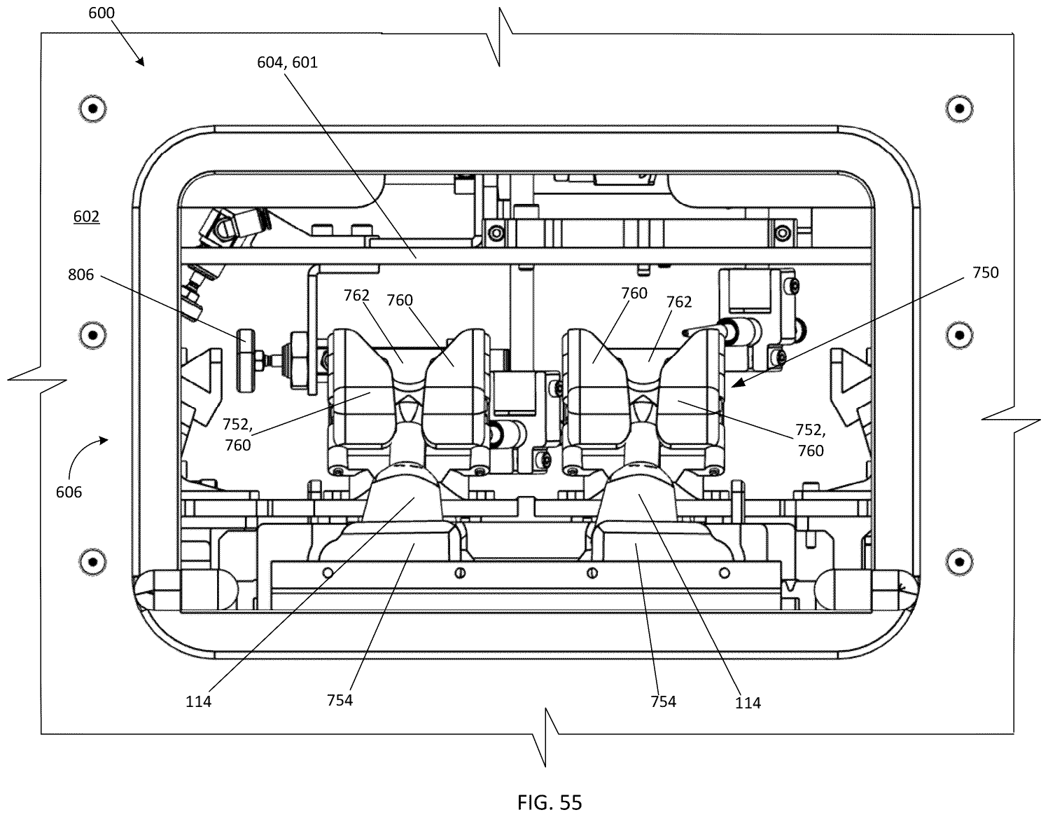

Referring to FIGS. 55-64, in some embodiments, in lieu of or in addition to holders 670, the administration device 600 can comprise holders 750. The holders 750 can be configured to restrain one or more subjects 114 against movement relative to the carousel 604. In the illustrated embodiment, each holder 750 is configured to restrain or hold two subjects 114. However, in other embodiments, each holder 750 can be configured to hold any number of subjects, for example, one, three, four, or five subjects, etc. The holders 750 can be coupled to the carousel 604 in a rotatable manner such that the holders 750 can rotate relative to the plurality of stations (e.g., stations 606, 608, 610, and 612 described previously). For example, as shown in FIG. 62, the holders 750 can be coupled to a movable portion 603 of the carousel 604 and the stations can be coupled to a stationary portion 601 of the carousel 604.

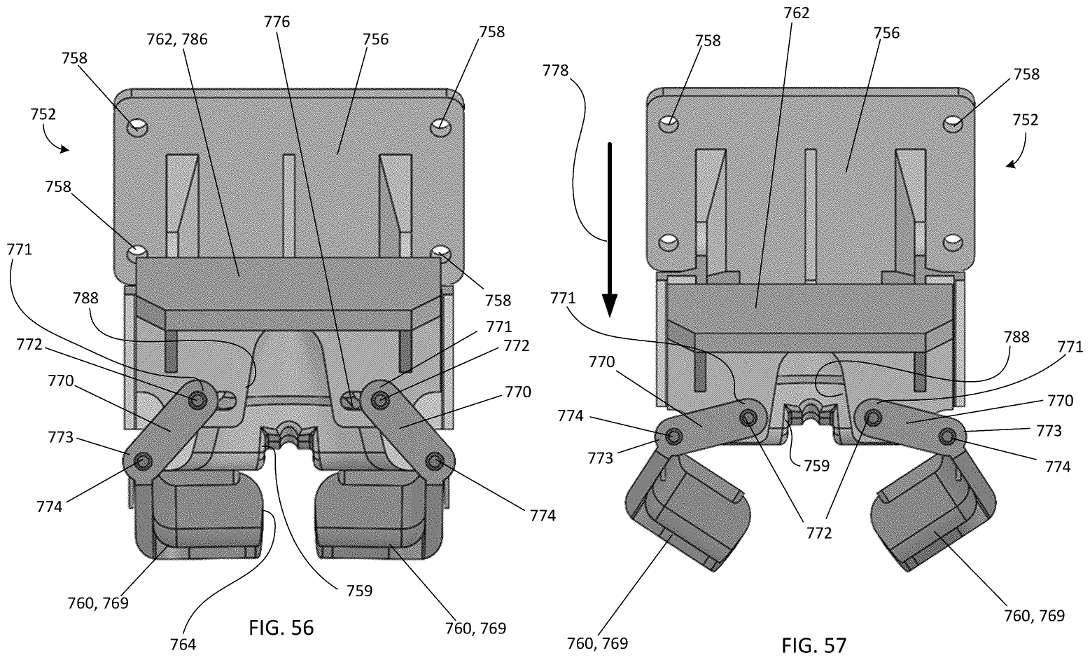

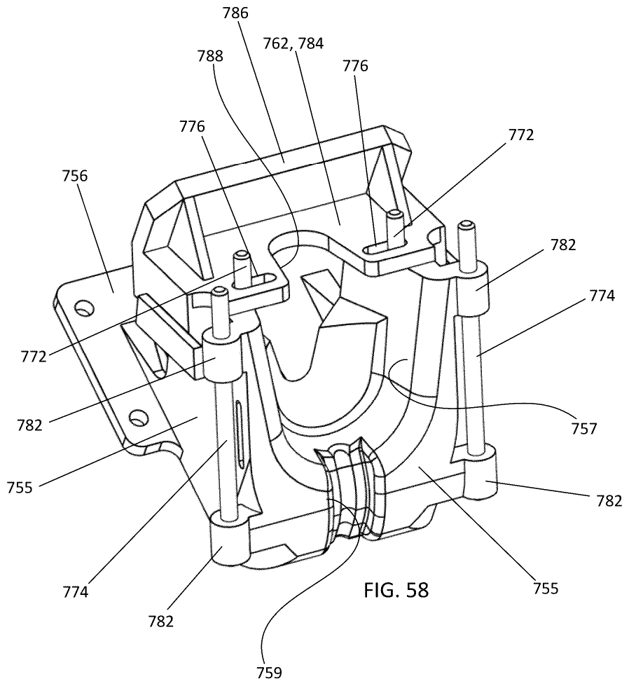

Each holder 750 can comprise one or more restraint portions 752, each configured to hold a subject 114, and one or more back supports 754 (similar to back supports 671 described previously). The back supports 754 can be pivotable between an open position and a closed position (shown in FIGS. 55-56). FIGS. 57-61 illustrate a single restraint portion 752. Referring to FIG. 57, each restraint portion 752 can comprise a base member 756 including a plurality of apertures 758 through which one or more fasteners can extend to couple the base member 756 to the movable portion 603 of the carousel. As best seen in FIG. 58, the base member 756 can comprise an extension portion 755 that defines a recess 757 (e.g., a U-shaped recess). The recess 757 is configured to receive and orient the subject's head (e.g., making sure the beak is facing forward into the device) when the subject is held by the restraint portion 752, as shown in FIG. 60. The extension portion 755 can further comprise one or more extension members 782 each including an inner bore through which a pivot member 774 can extend, as shown in FIG. 58.

In some embodiments, such as the embodiment shown in FIG. 58, the extension portion 755 can further comprise a cutout 759 extending through a thickness of the extension portion 755. The cutout 759 can be configured (e.g., sized and shaped) such that when a subject 114, e.g., a chick, is positioned within the restraint portion 752 the front side of the subject's neck is disposed within the cutout 759. Such a configuration can help stabilize the subject's neck, particularly during injections when the restraint portion 752 is located at the injection station 608. This can help ensure that the needle 688 is accurately positioned for injection into the back side of the subject's neck and also help mitigate or prevent injury to the subject 114 during injection. In the embodiment illustrated in FIGS. 56-61, the cutout 759 can comprise a scalloped edge portion, however, in other embodiments the cutout 759 can have any of various shapes configured to accurately position the subject's neck for injection.

Referring again to FIGS. 56-57, each restraint portion 752 can further comprise two clamp members 760 pivotably coupled to a movable member 762. The clamp members 760 can be movable between a first, or closed position (FIG. 56) and a second, or open position (FIG. 57). As shown in FIG. 56, when in the closed position, the clamp members 760 define a channel or passage 764 between them. The channel 764 can be sized such that a subject's 114 neck can rest within the channel 764 but such that the subject's head cannot pass through the channel 764. The subject's head is therefore restrained between the two clamp members 760 against movement relative to the restraint portion 752, see e.g., FIG. 60. As shown in FIG. 60, the channel 764 can have a first end portion 766 and a second end portion 768. The first end portion 766 can have an opening wider than the opening at the second end portion 768 such that the channel 764 tapers from the first end portion 766 to the second end portion 768. This configuration allows a user to quickly and easily position the subject's head within the restraint portion 752 such that the subject's neck is disposed within the channel 764.

Each clamp member 760 can comprise a hinge portion 770 extending from a main body 769 of the clamp member 760. Referring to FIGS. 56-57, each hinge portion 770 can have a first end portion 771 through which a fastener 772 can extend to couple the clamp member 760 to the movable member 762. The hinge portion 770 can have a second end portion 773 through which a pivot member 774 can extend to couple the clamp member 760 to the base member 756. The clamp member 760 can pivot around the pivot member 774 when actuated using the movable member 762.

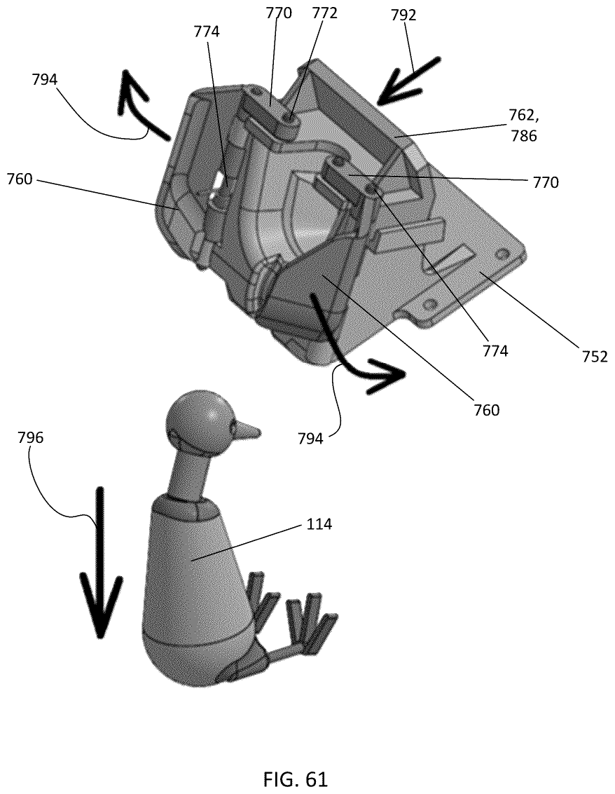

FIG. 58 shows the restraint portion 752 with the clamp members 760 removed for purposes of illustration. As shown, the movable member 762 can comprise one or more openings 776 in which the first fasteners 772 can be disposed. The openings 776 can be configured (e.g., sized and shaped) such that movement of the movable member 762 in a first direction (e.g., as indicated by arrow 778 in FIG. 57) causes the fastener 772 to slide within the opening 776 between a first position (corresponding with a closed position of the clamps 760, see FIG. 56) and a second position (corresponding with an open position of the clamps 760, see FIG. 57). This allows each clamp member 760 to pivot around its respective pivot member 774, as shown in FIG. 57. Movement of the clamp members 760 into the open position allows a subject 114 positioned within the restraint portion 752 to be released, as shown in FIG. 61.

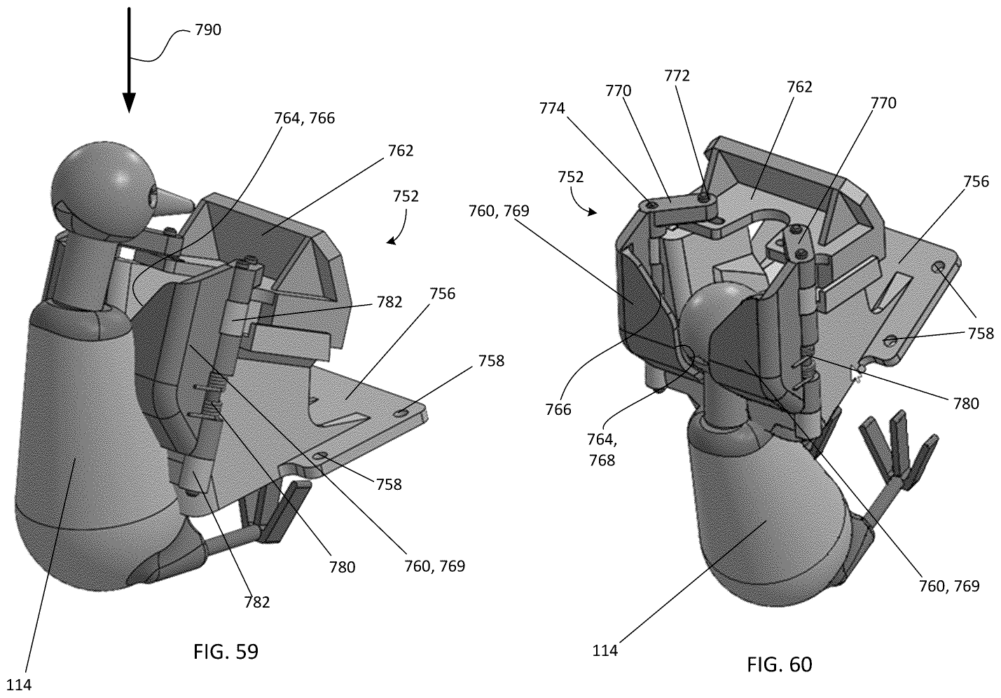

As shown in FIG. 59, the pivot members 774 can comprise one or more biasing members 780 (e.g., springs) configured to bias the clamp members 760 into the closed position. In some embodiments, such as the embodiment shown in FIGS. 55-64, the biasing members 780 can be torsion springs. In other embodiments, the biasing members 780 can be any of various other types of spring or mechanical means for biasing.

Referring to FIG. 58, the movable member 762 can comprise a main body 784 and a tab 786 that extends from the main body 784 at an angle. The main body 784 can comprise a cutout 788 disposed such that when a subject 114, e.g., a chick, is inserted into the restraint portion 752 the beak of the subject passes through the cutout 788.

Referring to FIGS. 59-60, during loading of the subject(s) 114 at the loading station 606, a user can insert the subject's head into the restraint portion 752 (e.g., in the direction of arrow 790) such that the subject's neck is disposed within the channel 764. The subject 114 will thereby be restrained against movement relative to the restraint portion 752 by the clamp members 760. In the embodiment illustrated in FIGS. 55-64, the clamp members 760 are biased to remain in the closed position unless the movable member 762 is actuated. However, in other embodiments, the clamp members 760 may be in the open position when a subject is disposed in the restraint portion 752, and can actuate automatically to the closed position in order to restrain the subject. In still other embodiments, the clamp members 760 can be manually actuated (e.g., by moving the clamp members by hand or by pressing a button).

As shown in FIG. 61, to release the subject 114 from the restraint portion 752, a force can be applied to the movable member 762 (e.g., in the direction of arrow 792), thereby moving the first end portion of the hinge members 770. This causes the clamp members 760 to pivot around the pivot members 774, as indicated by arrows 794, releasing the subject 114 from the device, as indicated by arrow 796.

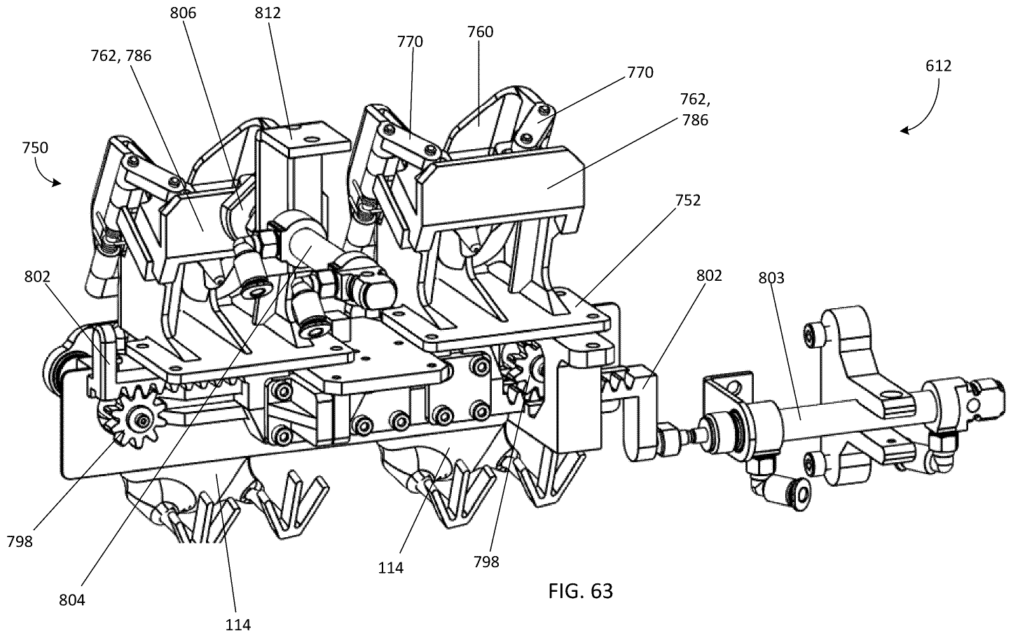

Once each subject 114 is disposed within a respective restraint portion 752, each back support 754 can rotate into a closed position (see e.g., FIG. 62) to further restrain the subject against movement relative to the holder 750. As shown in FIG. 63, each back support 754 can be coupled to a toothed component or gear 798 via an arm 800 (FIG. 62). The gear 798 can be actuated by one or more correspondingly toothed linear members 802 such that the back support 754 can rotate or pivot between an open position and the closed position. As shown in FIG. 63, the toothed linear members 802 can be coupled to an actuator 803 (e.g., a pneumatic piston), which can actuate the members 802 to open and close the back support 754.

Once the subject(s) 114 have been loaded into the holder 750 via the loading station 606, the movable portion 603 of the carousel 604 can rotate, moving the subjects from the loading station 606 to the injection station 608. In some embodiments, the loading station 606 can comprise a sensor (e.g., a weight sensor) configured to determine whether one or more subjects 114 have been loaded into the holder 750. In some embodiments, once at least one subject 114 has been loaded, the administration device 600 will automatically rotate the holder 750 to the next station, for example, the injection station 608. In some embodiments, the loading station can comprise one or more sensors configured to determine whether the user's hands are within the loading station. The administration device 600 will not automatically rotate until the user's hands have been removed from the loading station 606.

The movable portion 603 (FIG. 62) of the carousel 604 can rotate the holder 750 through the loading station 606, injection station 608, spraying station 610, and release station 612 in sequence, as described previously with respect to holder 670.

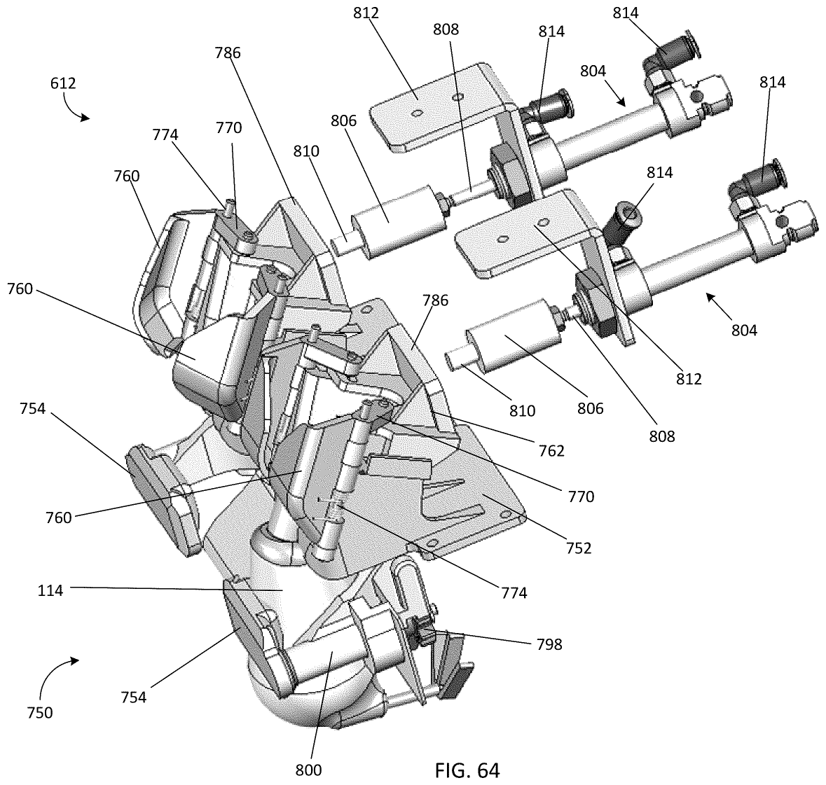

Referring to FIGS. 63-64, the release station 612 of the administration device 600 can comprise one or more actuators 804 (e.g., pneumatically actuated pistons) configured to actuate the movable member(s) 762 of the holder 750 by applying force to a respective tab 786. As shown in FIG. 64, in embodiments wherein the holder comprises two restraint portions 752, the release station 612 can comprise two corresponding actuators 804. In embodiments wherein the holder 750 comprises one, or more than two restraint portions 752, the release station 612 can comprise a corresponding number of actuators 804. For purposes of illustration, FIG. 63 shows only a single actuator 804.

In the embodiment of FIG. 64, each actuator 804 can comprise a head portion 806 coupled to the piston 808 of the actuator 804. In the illustrated embodiment, the head portion 806 is a cylindrical member including an optional cylindrical extension 810 having a diameter smaller than a diameter of the head portion. In other embodiments, the head portion 806 and/or the cylindrical extension 810 can have any of various shapes. For example, FIG. 63 illustrates an embodiment wherein the head portion 806 is a flat, substantially circular member. Each actuator 804 can be coupled to the stationary portion 601 of the carousel 604, for example, using a bracket 812, such as an L-bracket. In the embodiment shown in FIG. 64, each actuator 804 comprises one or more inlets and outlets 814 configured to allow the actuator 804 to be actuated pneumatically. In other embodiments, the actuators 804 and/or the movable members 762 can be actuated using other means, e.g., electrically (such as by a microprocessor), manually (e.g., by pressing a button, flipping a switch, or turning a lever), magnetically, and/or hydraulically.

Referring to FIG. 64, once the holder 750 is positioned at the release station 612, the one or more toothed linear members 802 (FIG. 63) can be actuated to actuate the corresponding gears 798 (FIG. 63) to pivot the back supports 754 into the open position. The actuators 804 can then be actuated such that they apply force to the tabs 786, thereby moving the movable members 762 and opening the clamp members 760 to release the subject(s) 114. The subjects 114 can slide down the ramp 726 as described previously with respect to FIG. 54.

In view of the many possible embodiments to which the principles of the disclosure may be applied, it should be recognized that the illustrated embodiments are only preferred examples and should not be taken as limiting the scope of the disclosure. Rather, the scope is defined by the following claims. We therefore claim all that comes within the scope and spirit of these claims.

We claim:

1. A holder for a medicament administration device, comprising:
a base member;
a movable member disposed on the base member such that the movable member can move relative to the base member between a first position and a second position; and
two or more clamp members each coupled to the base member and to the movable member, the clamp members being pivotable between a closed position corresponding with the first position of the movable member and an open position corresponding with the second position of the movable member, wherein when in the closed position the clamp members are configured to engage a head of a subject to restrain the subject against movement relative to the holder.

2. The holder of claim 1, wherein when in the closed position the clamp members define a channel between them, the channel sized such that a subject's neck can be disposed within the channel.

3. The holder of claim 2, wherein the channel has a first end portion and a second end portion and a width of the channel tapers from a first, greater width at the first end portion to a second, narrower width at the second end portion.

4. The holder of claim 1, wherein the clamp members are pivotably coupled to the base member via respective pivot members extending through the clamp member and the base member.

5. The holder of claim 4, wherein the base member comprises one or more extension members each having an inner bore, and wherein the pivot members extend through the inner bore of at least one extension member.

6. The holder of claim 1, wherein the clamp members are coupled to the movable member via respective fasteners extending through the clamp members and into one or more channels disposed in the movable member, and wherein movement of the movable member between the first and second positions causes corresponding movement of the fasteners within the channels.

7. The holder of claim 1, further comprising a biasing member configured to bias the clamp members into the closed position.

8. The holder of claim 1, wherein the base member comprises an extension portion defining a recess, the recess configured to receive and orient the subject's head when the subject is positioned within the holder.

9. The holder of claim 8, wherein the recess comprises a U-shape.

10. The holder of claim 1, wherein the movable member comprises a main body and a tab extending from the main body at an angle.

11. The holder of claim 1, wherein the movable member comprises a U-shaped cutout.

12. The holder of claim 1, further comprising one or more back supports configured to support a back of the subject to help restrain the subject against movement relative to the holder.

13. The holder of claim 12, wherein the back supports are pivotable between an open position and a closed position.

14. A method of using the holder of claim 1, comprising:
positioning the subject such that the subject's neck is aligned with a first end portion of a channel defined by the two or more clamp members; and
inserting the subject's neck into the channel such that the subject's head is restrained by the two or more clamp members, thereby restraining the subject against movement relative to the holder.

15. The method of claim 14, further comprising:
applying a force to the movable member causing the movable member to move from the first position to the second position, thereby pivoting the two or more clamp members into the open position such that the subject is released from the holder.

16. The method of claim 15, wherein applying a force to the movable member comprises actuating a pneumatic piston, wherein a head portion of the piston contacts the movable member to provide the force.

17. A medicament administration device, comprising:
a plurality of stations including, at least, a loading station configured to allow insertion of a subject into the administration device, and a release station configured to automatically release the subject from the administration device;
a carousel having a stationary portion on which the plurality of stations is disposed, and a movable portion that is movable relative to the stationary portion; and
a holder coupled to the movable portion of the carousel and configured to engage a subject and rotate the subject between the plurality of stations, the holder comprising:
a base member,
a movable member disposed on the base member such that the movable member can move relative to the base member between a first position and a second position, and
two or more clamp members coupled to the base member and to the movable member, the clamp members being pivotable between a closed position corresponding with the first position of the movable member and an open position corresponding with the second position of the movable member, wherein when in the closed position the clamp members are configured to engage the subject to restrain the subject against movement relative to the holder.

18. The administration device of claim 17, further comprising:
an injection station configured to automatically administer a medicament to one or more subjects using one or more injectors; and
a spray station configured to automatically administer a medicament to one or more subjects using one or more spray nozzles.

19. The administration device of claim 17, wherein the loading station comprises a sensor configured to determine whether a hand of a user is present within the loading station.

20. The administration device of claim 17, wherein the holder further comprises one or more back supports configured to support a back of the subject to help restrain the subject against movement relative to the holder.

21. The administration device of claim 20, wherein the loading station and the release station comprise an actuator configured to move a toothed linear member operatively engaged with the back support in order to pivot the back support between an open position and a closed position.

22. The administration device of claim 17, wherein the one or more subjects are day-old chicks.

23. A method, comprising:
providing an administration device according to claim 18; and
inserting a subject into the holder by inserting the subject's neck between a first set of the two or more clamp members;
rotating the subject to the injection station;
inserting a needle coupled to a respective injector of the one or more injectors to a selected depth within the subject;
automatically administering a dose of medicament when the needle reaches the selected depth;
rotating the subject to the spraying station; and
automatically administering a dose of medicament to the subject via the one or more spray nozzles.
